(19) United States
(12) Patent Application Publication
(10) Pub. No.: US 2024/0308942 A1
(43) Pub. Date: Sep. 19, 2024

(54) PROCESS FOR THE ENERGY-EFFICIENT PREPARATION OF ALKALI METAL ALCOHOLATES

(71) Applicant: EVONIK OPERATIONS GMBH, Essen (DE)

(72) Inventors: Dirk Roettger, Köln (DE); Sebastian REIMANN, Wesseling (DE); Niklas PAUL, Marl (DE); Armin RIX, Marl (DE); Philip ZITZEWITZ, Haltern am See (DE); Moritz FCHRÖDER, Münster (DE)

(73) Assignee: EVONIK OPERATIONS GMBH, Essen (DE)

(21) Appl. No.: 18/275,469

(22) PCT Filed: Jan. 27, 2022
(86) PCT No.: PCT/EP2022/051869

§ 371 Date: Aug. 2, 2023

(30) Foreign Application Priority Data
Feb. 5, 2021 (EP) 21155473.8

(51) Int. Cl. C07C 29/70; C07C 29/80

(52) U.S. Cl. CPC C07C 29/70; C07C 29/80

(57) ABSTRACT

The present invention relates to a process for preparing alkali metal alcoholates in counter flow by way of reactive rectification, the alkali metal being selected from sodium and potassium. The process is carried out in at least one reaction column and at least one rectification column. The process according to the invention is characterized in that the heating steam used for operating the columns and thus for carrying out the process condenses, and that the energy is used in the condensed water.

The present invention relates to a process for producing alkali metal alkoxides in countercurrent by reactive rectification, wherein the alkali metal is selected from sodium and potassium. The process is carried out in at least one reaction column and at least one rectification column. The process is characterized in that the heating vapour utilized for operating the column and thus for carrying out the process is condensed and the energy in the condensate obtained is used.

### 1. BACKGROUND OF THE INVENTION

The production of alkali metal alkoxides is an important industrial process.

Alkali metal alkoxides are used as strong bases in the synthesis of numerous chemicals, for example in the production of pharmaceutical or agrochemical active ingredients. Alkali metal alkoxides are also used as catalysts in transesterification and amidation reactions.

Alkali metal alkoxides (MOR) are produced by reactive distillation of alkali metal hydroxides (MOH) and alcohols (ROH) in a countercurrent distillation column, wherein the water of reaction formed according to the following reaction <1> is removed with the distillate.

MOH+ROH [symbol] MOR+H₂O

Such a process principle is described, for example, in U.S. Pat. No. 2,877,274 A, wherein aqueous alkali metal hydroxide solution and gaseous methanol are driven in countercurrent into a rectification column. This process is described again in generally unchanged form in WO 01/42178 A1.

Similar processes, which, however, additionally employ an entraining agent such as benzene, are described in GB 377,631 A and U.S. Pat. No. 1,910,331 A. The entraining agent here serves to separate water from the water-soluble alcohol, and the condensate is subjected to phase separation in order to separate off the water of reaction.

Correspondingly, DE 96 89 03 C describes a process for continuous production of alkali metal alkoxides in a reaction column, wherein the water-alcohol mixture withdrawn at the top of the column is condensed and then subjected to a phase separation. The aqueous phase is discarded and the alcoholic phase is returned to the top of the column together with the fresh alcohol. EP 0 299 577 A2 describes a similar process, wherein the water in the condensate is separated off with the aid of a membrane.

The most industrially important alkali metal alkoxides are those of sodium and potassium, especially the methoxides and ethoxides. Their synthesis is frequently described in the prior art, for example in EP 1 997 794 A1.

The syntheses of alkali metal alkoxides by reactive rectification described in the prior art typically afford vapours comprising the employed alcohol and water. It is advantageous for economic reasons to reuse the alcohol comprised in the vapours as a reactant in the reactive distillation. The vapours are therefore typically supplied to a rectification column and the alcohol present therein is separated off (described for example in GB 737 453 A and U.S. Pat. No. 4,566,947 A). The thus recovered alcohol is then supplied to the reactive distillation as a reactant for example. Alternatively or in addition a portion of the alcohol vapour may be utilized for heating the rectification column (described in WO 2010/097318 A1). However, this requires that the vapour be compressed in order to achieve the temperature level required for heating the rectification column.

The energy required for operating the process is normally introduced by means of heating vapour into the process of the invention, for example via heat exchangers such as vaporizers at the bottom of the column or for heating the alcohol or alkali metal hydroxide solution. In the transfer of energy, the heating vapour condenses and the resulting liquid phase, namely the heating vapour condensate, frequently still contains considerable amounts of residual heat. This results in the following disadvantages: Firstly, it is energetically undesirable for this energy to be allowed to dissipate unutilized. In addition, condensates are usually collected in a condensate network in an industrial context (chemical facilities and technology parks). When this network is fed from different plants, the condensates introduced differ in terms of temperature and pressure. In order to prevent pressure pulses in the condensate network, each plant connected to the network therefore has to actively cool the corresponding condensate, which in turn requires additional energy expenditure.

It was accordingly an object of the present invention to provide a process for producing alkali metal alkoxides which alleviates the abovementioned disadvantages and in particular allows efficient utilization of the energy in the heating vapour condensates. Apart from the advantageous saving of energy, this should also allow the condensate to be set to particular pressure and temperature values easily and energy-efficiently.

A process which achieves this object has now surprisingly been found.

### 2. BRIEF SUMMARY OF THE INVENTION

The present invention accordingly provides a process according to claim 1.

### 3. FIGURES

FIG. 1 shows a process which is not according to the invention for producing alkali metal alkoxides using various heat exchangers.

Such a plant comprises a reaction column RR_{A}<1A>. At the top of the reaction column RR_{A}<1A>, an aqueous NaOH solution is introduced as feed stream S_{AE2}<1A2>. As an alternative, it is also possible to introduce a methanolic NaOH or KOH solution as feed stream S_{AE2}<1A2> in order then to produce the corresponding sodium or potassium methoxide. Energy can optionally be introduced into the feed stream S_{AE2}<1A2> via a heat exchanger WT<WA5>, for example in order to preheat this feed stream. Above the bottom of the reaction column RR_{A}<1A>, methanol is introduced in vapour form as feed stream S_{AE1}<1A1>. Energy can optionally be introduced into the feed stream S_{AE1}<1A1> via a heat exchanger WT<WA4>, for example in order to preheat this feed stream. The reaction of the two feed streams S_{AE1}<1A1> and S_{AE2}<1A2> results in formation in the reaction column RR_{A}<1A> of a crude product mixture RP_{A}<1A3> which comprises NaOCH₃, water, CH₃OH, NaOH when S_{AE2}<1A2> comprises NaOH. When S_{AE2}<1A2> comprises KOH, a crude product mixture RP_{A}<1A3> comprising KOCH₃, water, CH₃OH and KOH is obtained.

At the bottom of the reaction column RR_{A}<1A>, the product stream S_{AS}<1A4> which comprises sodium methoxide or potassium methoxide dissolved in methanol is obtained. The concentration of the sodium or potassium methoxide solution S_{AS}<1A4> can be set to the desired value by means of the optional bottom vaporizer VS_{RRA}<WA1> at the bottom of the column RR_{A}<1A>. Energy can optionally be transferred by means of the optional bottom vaporizer VS_{RRA}<WA2> to part of S_{AS}<1A4> which is recirculated to the reaction column RR_{A}<1A>. VS_{RRA}<WA2> also serves, in particular, for starting up the column RR_{A}<1A>. The optional intermediate vaporizer VZ_{RRA}<WA3> offers an opportunity to transfer energy to the crude product mixture RP_{A}<1A3>. This is taken off from the column RR_{A}<1A> as stream S_{RRAZ}<1A7>, the stream S_{RRAZ}<1A7> is heated in the intermediate vaporizer VZ_{RRA}<WA3> and then returned to the column RR_{A}<1A>.

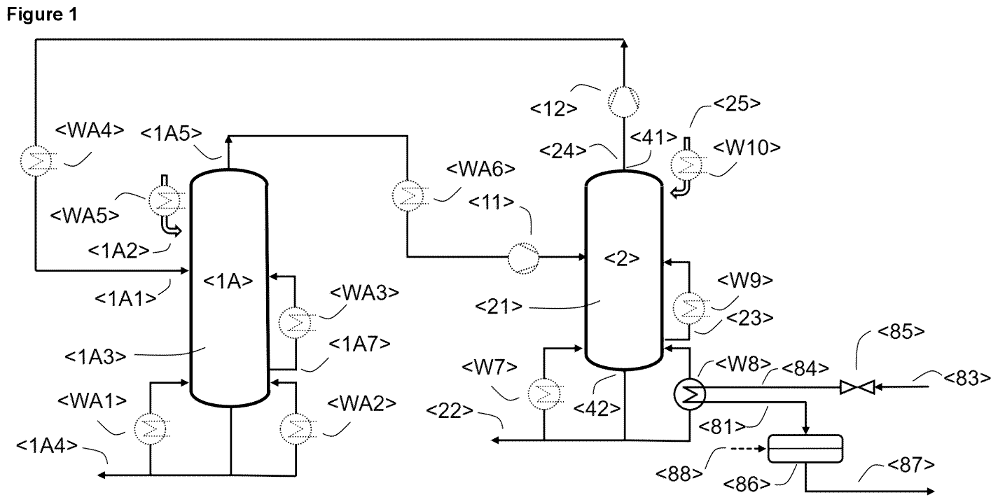

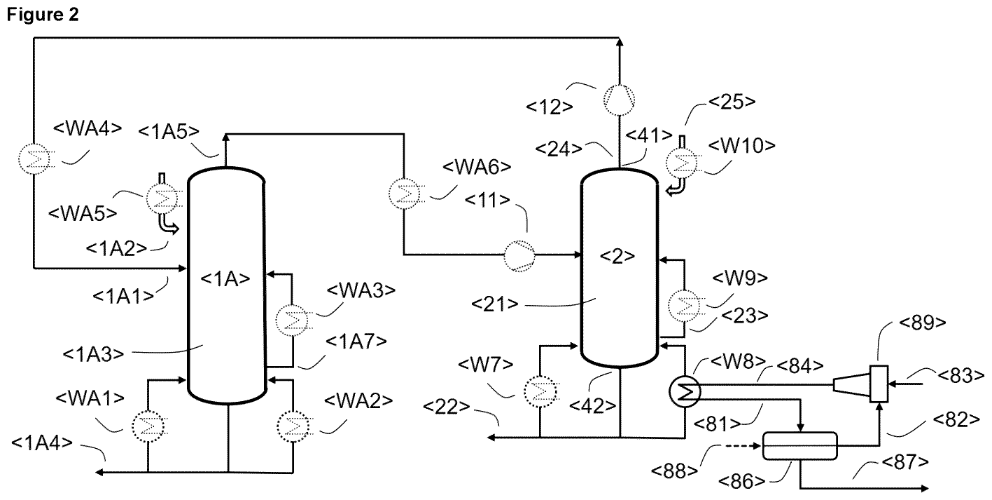

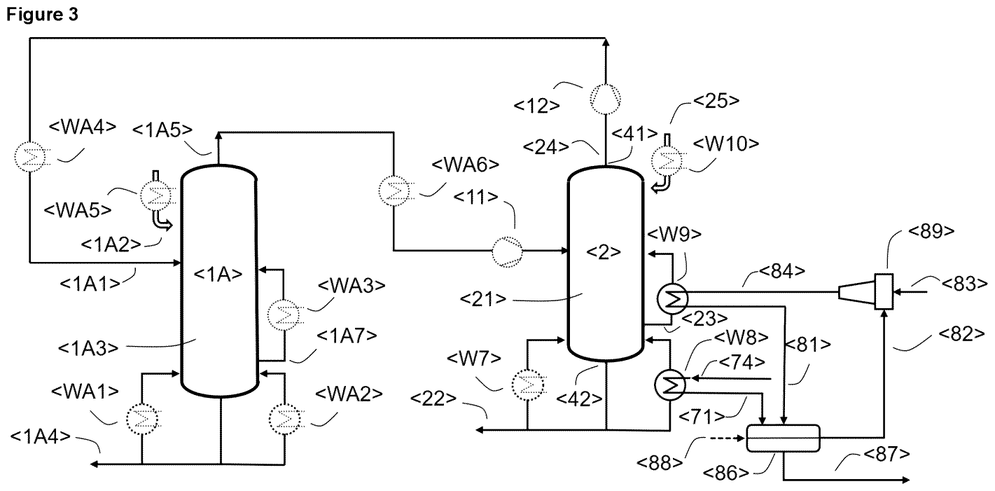

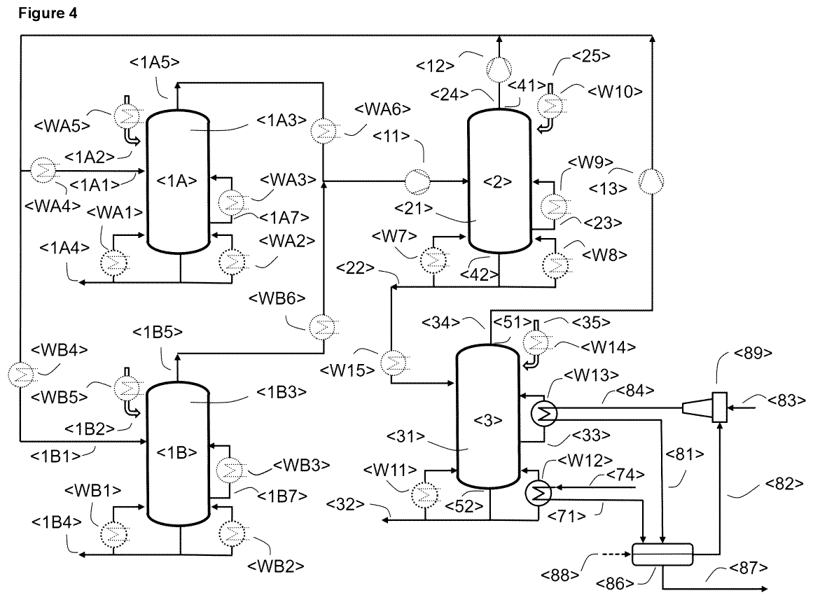

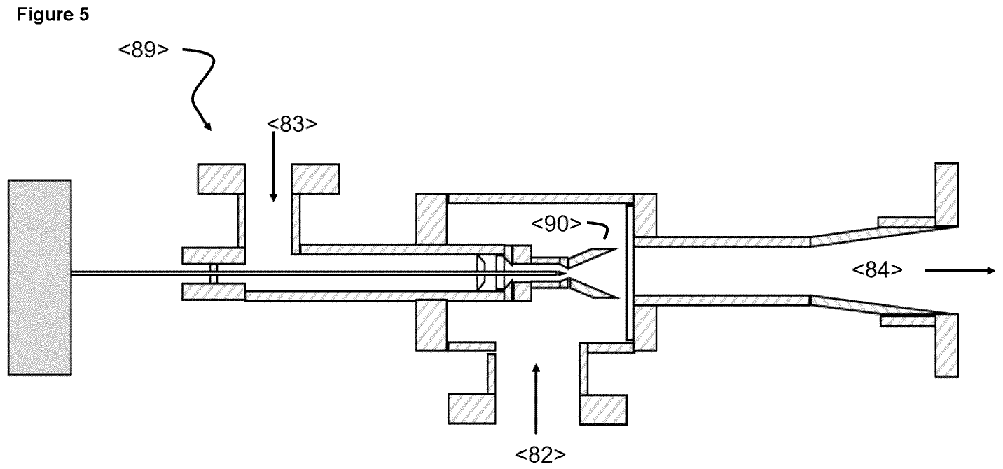

A vapour stream S_{AB}<1A5> is taken off at the top of the reaction column RR_{A}<1A>. Part of the vapour stream S_{AB}<1A5> can be condensed in a condenser K_{RRA} at the top of the reaction column RR_{A}<1A> and returned in liquid form as runback to the top of the reaction column RR_{A}<1A>. However, this embodiment which is not shown in FIG. 1, namely the setting of the runback at the top of the reaction column RR_{A}<1A>, is optional.

The vapour stream S_{AB}<1A5> is optionally conveyed via a heat exchanger WT<WA6> and via a compressor VD₃₁<11> to a rectification column RD_{A}<2>. A mixture G_{A}<21> which comprises water and methanol is obtained in the rectification column RD_{A}<2> and is separated in RD_{A}<2> into a vapour stream S_{DAB}<24> comprising methanol and a bottom stream S_{DAS}<22> comprising water and ROH. At the bottom of the column RD_{A}<2>, the vapour stream S_{DAS}<22> is discharged at an offtake point E_{AK}<42> and can partly be recirculated again via the optional bottom vaporizer VS_{A}<W7> or the bottom vaporizer VS_{A}<W8> to the column RD_{A}<2>.

At the top of the rectification column RD_{A}<2>, methanol vapour S_{DAB}<24> is discharged at an offtake point E_{AK}<41>. This is then optionally conveyed via the compressor <12> and recirculated to the reaction column RR_{A}<1A> where it is introduced as feed stream S_{AE1}<1A1>. The compressor <12> can also be used instead of the compressor VD₃₁<11>.

The intermediate vaporizer VZ_{A}<W9> offers a possibility of transferring energy to the mixture G_{A}<21>. This is taken off from the column RD_{A}<2> as stream S_{AZ}<23>, the stream S_{AZ}<23> is heated in the intermediate vaporizer VZ_{A}<W9> and then recirculated to the column RD_{A}<2>.

A stream of fresh methanol <25> can be supplied to the system via the rectification column RD_{A}<2>. Energy can be introduced into this stream via a heat exchanger WT<W10>. The fresh methanol can be introduced directly into the rectification column RD_{A}<2>.

Energy is transferred by means of the bottom vaporizer VS_{A}<W8> to part of S_{DAS}<22> which is recirculated to the rectification column RD_{A}<1A>. The bottom vaporizer VS_{A}<W8> here also serves, in particular, for starting up the column RD_{A}<2>. FIG. 1 shows, for the bottom vaporizer VS_{A}<W8>, the heating by means of heating vapour H₁<84> according to the prior art. Accordingly, energy is transferred from heating vapour H₁<84> via the bottom vaporizer VS_{A}<W8> to recirculated S_{DAS}<22> and thus ultimately made available for separation of the mixture G_{A}<21> by distillation.

The condensation temperature of the heating vapour H₁<84> has to be set according to the required heating power for the distillation. Since the temperature or the pressure of the heating vapour H₃<83> available in industrial plants is usually different from the values desired in the specific case, H₃<83> firstly has to be depressurized to H₁<84>, which occurs by means of a regulating valve <85>. This occurs with dissipation of parts of the energy present in the heating vapour H₃<83>.

As a result of the transfer of energy from H₁<84> to S_{DAS}<22> in the bottom vaporizer VS_{A}<W8>, H₁<84> condenses at least partially to form the condensate K₁<81> which is collected in a condensate vessel <86> where condensates <88> from other heat exchangers of the plant as shown in FIG. 1 or even other plants (e.g. in integrated sites) are optionally collected. The collected condensates then have to be disposed of as condensate stream <87> or can, for instance, be fed into the condensate network in chemical facilities. As a result, not only is part of the energy present in the condensates discarded unutilized, but it is in many cases also necessary to set these condensates to a particular temperature with additional energy consumption in order to avoid pressure pulses in the condensate network.

This problem arises not only in the case where the heating vapour H₁<84> heats the bottom vaporizer VS_{A}<W8>. In plants which correspond to that in FIG. 1, but in which energy of heating vapour H₃<83> is instead to be introduced via or in addition to the bottom vaporizer VS_{A}<W8> via another heat exchanger, the same problem arises.

FIG. 2 shows an embodiment of the process of the invention. The plant corresponds to that described in FIG. 1. The characteristic feature of the process of the invention is illustrated with the aid of the bottom vaporizer VS_{A}<W8>. However, the heating vapour H₁ can, as an alternative or in addition, also be conveyed to one of the other heat exchangers WT<WA1> to <WA6>, <W7>, <W9>, <W10>.

According to the procedure in the prior art, energy is also transferred from heating vapour H₁<84> to S_{DAS}<22> via the bottom vaporizer VS_{A}<W8> and thus ultimately made available to the separation of the mixture G_{A}<21> by distillation. This gives a heating vapour condensate K₁<81> which is collected and depressurized in the condensate vessel <86>, optionally together with other condensates <88>. The condensates <88> can arise from heating by means of one or the other heat exchangers WT<WA1> to <WA6>, <W7>, <W9>, <W10>, when the plant comprises such a heat exchanger, or, when the process of the invention is carried out in the vicinity of another plant in which heating vapour condensate is obtained, can be conveyed from such a plant into the condensate vessel <86>. The critical difference from the processes of the prior art is that at least part of the heating vapour condensate K₁<81> is depressurized and a heating vapour H₂<82> which has a pressure p₂ which is lower than the pressure p₁ of H₁<84> is thus obtained. The resultant heating vapour H₂<82> is then mixed with fresh heating vapour H₃<83> having a pressure p₃ which is > p₂, where, in the embodiment of FIG. 2, p₃ is also > p₁. In this way, the heating vapour/condensate/heating vapour circuit of H₁<84>/K₁<81>/H₂<82> is completed and new heating vapour H₄, which is identical to the heating vapour H₁<84> with the desired pressure (and thus the desired temperature) is obtained and can be used again in the next round as heating vapour H₁<84> for heating the bottom vaporizer VS_{A}<W8>.

Compared to the processes of the prior art, the following advantages are obtained thereby:
A higher proportion of the energy in the condensate K₁ is integrated into the process.
As a result of the energy in the condensate K₁ being utilized, the condensate K₁ has to be cooled to a lesser degree before it is fed into the condensate network in order to decrease the probability of pressure pulses in the condensate network. This saves additional cooling energy.

FIG. 3 shows a further embodiment of the process of the invention. This corresponds to the embodiment described in FIG. 2 except that energy is transferred from H₁<84> to the mixture G_{A}<21> in the intermediate vaporizer VZ_{A}<W9> on the rectification column RD_{A}<2>. In addition, the condensate vessel <86> is also supplied with hot steam condensate <71> which is obtained by condensation of heating vapour <74> during operation of the bottom vaporizer VS_{A}<W8>.

FIG. 4 shows a further embodiment of the process of the invention. This comprises the reaction column RR_{A}<1A> described in FIG. 3 and also the rectification column RD_{A}<2>. In addition, the embodiment of FIG. 4 has the following features:

Apart from the reaction column RR_{A}<1A>, the plant used in the process comprises a further reaction column RR_{B}<1B> into which an aqueous KOH solution is introduced as feed stream S_{BE2}<1B2>. It is also possible to employ the aqueous or alcoholic solution of NaOH, but S_{BE2}<1B2> is advantageously selected so that the alkali metal present in this stream differs from that in S_{AE2}<1A2>. Energy can optionally be introduced into the feed stream S_{BE2}<1B2> via a heat exchanger WT<WB5>, for example in order to preheat this feed stream. When S_{BE2}<1B2> comprises NaOH, S_{BE2}<1B2> is reacted in RR_{B}<1B> with a stream S_{BE1}<1B1> which comprises CH₃OH and has optionally been preheated by means of the heat exchanger WT<WB4> to give a crude product mixture RP_{B}<1B3> comprising NaOCH₃, water, CH₃OH, NaOH. When S_{BE2}<1B2> comprises KOH, a crude product mixture RP_{B}<1B3> comprising KOCH₃, water, CH₃OH and KOH is obtained. In this embodiment, ROH is thus methanol.

At the bottom of the reaction column RR_{B}<1B>, the product stream S_{BS}<1B4> comprising sodium methoxide or potassium methoxide dissolved in methanol is obtained. The concentration of the sodium or potassium methoxide solution S_{BS}<1A4> can be set to the desired value using the optional bottom vaporizer VS_{RRB}<WB1> at the bottom of the column RR_{B}<1B>. Energy can optionally be transferred by means of the optional bottom vaporizer VS_{RRB}<WB2> to part of S_{B}S <1B4> which is recirculated to the reaction column RR_{B}<1B>. VS_{RRB}<WB2> here also serves, in particular, for starting up the column RR_{B}<1B>. The optional intermediate vaporizer VZ_{RRB}<WB3> here offers an opportunity for transferring energy to the crude product mixture RP_{B}<1B3>. This is taken off from the column RR_{B}<1B> as stream S_{RRBZ}<1B7>, the latter is heated in the optional intermediate vaporizer VZ_{RRB}<WB3> and then recirculated to the column RR_{B}<1B>.

At the top of the reaction column RR_{A}<1B>, a vapour stream S_{BB}<1B5> is taken off. Part of the vapour stream S_{AB}<1B5> can be condensed in a condenser at the top of the reaction column RR_{B}<1B> and returned in liquid form as runback to the top of the reaction column RR_{B}<1B>. However, this embodiment which is not shown in FIG. 4, i.e. setting of the runback at the top of the reaction column RR_{B}<1B>, is optional.

The vapour stream S_{BB}<1B5> is optionally conveyed via a heat exchanger WT<WB6>. S_{BB}<1B5> is combined with the stream S_{AB}<1A5> and then, after the combined stream has been conveyed via a compressor VD₃₁<11>, fed to a rectification column RD_{A}<2>. The two streams S_{AB}<1A5> and S_{BB}<1B5> can also be conveyed separately into the rectification column RD_{A}<2> and only be combined in the latter.

The distillation separation of the mixture G_{A}<21> into a vapour stream S_{DAB}<24> comprising ROH and a bottom stream S_{DAS}<22> comprising water and ROH takes place in the rectification column RD_{A}<2>. The bottom stream S_{DAS}<22> is discharged at an offtake point E_{AK}<42> at the bottom of the column RD_{A}<2> and can be partly recirculated via the optional bottom vaporizer VS_{A}<W7> or the bottom vaporizer VS_{A}<W8> at the bottom of the column RD_{A}<2> back to the column RD_{A}<2>. The bottom vaporizer VS_{A}<W8> here also serves, in particular, for starting up the column RD_{A}<2>.

At the top of the rectification column RD_{A}<2>, methanol vapour S_{DAB}<24> is discharged at an offtake point E_{AK}<41>. This is then optionally conveyed through the compressor <12> and recirculated to the reaction column RR_{A}<1A> where it is used as feed stream S_{AE1}<1A1> or S_{BE1}<1B1>. The compressor <12> can also be used instead of the compressor VD₃₁<11>.

The intermediate vaporizer VZ_{A}<W9> offers an opportunity for transferring energy to the mixture G_{A}<21>. This is taken off as stream S_{AZ}<23> from the column RD_{A}<2>, the stream S_{AZ}<23> is heated in the intermediate vaporizer VZ_{A}<W9> and then recirculated to the column RD_{A}<2>.

A stream of fresh methanol <25> can be supplied to the system via the rectification column RD_{A}<2>. Energy can be introduced into this via a heat exchanger WT<W10>. The fresh methanol <25> can be introduced directly into the rectification column RD_{A}<2>.

S_{DAS}<22> is optionally conveyed via a heat exchanger WT<W15> to a rectification column RD_{X}<3>. A mixture G_{X}<31> comprising water and methanol is thereby formed in RD_{X}<3>.

The distillative separation of the mixture G_{X}<31> into a vapour stream S_{XB}<34> comprising ROH and a bottom stream S_{XS}<32> comprising water and ROH takes place in the rectification column RD_{X}<3>. The bottom stream S_{XS}<32> is taken off at the offtake point E_{XS}<52> and discharged. At the bottom of the column RD_{X}<3>, the bottom stream S_{XS}<32> can partly be recirculated either via the optional bottom vaporizer V_{SX}<W11> or the optional bottom vaporizer V_{SX}<W12> to the column RD_{X}<3>. Energy can optionally be transferred by the optional bottom vaporizer VS_{X}<WA2> to part of S_{XS}<32> which is recirculated to RD_{X}<3>. VZ_{X}<W12> here also serves, in particular, for starting up the column RD_{X}<3>.

At the top of the rectification column RD_{X}<3>, methanol vapour S_{XB}<34> is taken off at an offtake point E_{XK}<51> and discharged. This vapour is then optionally conveyed via the compressor <13> and mixed with S_{DAB}<24> and then recirculated to the reaction columns RR_{A}<1A> and RRB <1B> and used as feed stream S_{AE1}<1A1> or S_{BE1}<1B1>.

The intermediate vaporizer VZ_{X}<W13> offers an opportunity for transferring energy to the mixture G_{X}<31>. This mixture is taken off from the column RD_{X}<3> as stream S_{XZ}<33>, the latter is heated in VZ_{X}<W13> and then recirculated to the column RD_{X}<3>.

A stream of fresh methanol <35> can be fed to the system via the rectification column RD_{X}<3>. Energy can be introduced into this stream by means of a heat exchanger WT<W14>. The fresh methanol <35> can here by introduced directly into the rectification column RD_{X}<3>.

Energy of heating vapour H₁<84> is transferred via the intermediate vaporizer VZ_{X}<W13> to S_{XS}<32> and thus ultimately made available for the distillative separation of the mixture G_{X}<31>. This results in a heating vapour condensate K₁<81> which can be collected in a condensate vessel <86>, optionally together with other condensates <88>. The condensates <88> can arise from the heating by means of one of the other heat exchangers <WA1> to <WA6>, <W7>, <W9>, <W10>, if the plant has such a heat exchanger, or, when the process of the invention is carried out in the vicinity of a corresponding plant in which the heating vapour condensate is obtained, be conveyed from such a plant into the condensate vessel <86> (stream <88>). In FIG. 4, a condensate <71> which arises from the heating of the bottom vaporizer V_{SX}<W12> by means of heating vapour <74> is likewise collected in the condensate vessel <86>.

At least part of the condensate in the collection vessel <86> is depressurized and vaporized to give further heating vapour H₂<82>, the pressure p₂ of which is less than the pressure p₁ of H₁<84>. The resulting heating vapour H₂<82> is then mixed with fresh heating vapour H₃<83>, the pressure p₃ of which is > p₁ and p₃ is thus also > p₂. The heating vapour/condensate/heating vapour circuit of H₁<84>/K₁<81>/H₂<82> is thus completed and new heating vapour H₄ is obtained which is identical to the heating vapour H₁<84> with the desired pressure (and thus the desired temperature) and can be reused in the fresh round as heating vapour H₁<84> for heating the intermediate vaporizer VZ_{X}<W13>.

FIG. 5 shows the schematic structure of a vapour ejector DS <89>. The driving vapour H₃<83> is here the heating vapour, in particular the intermediate pressure vapour from the vapour network. The suction vapour H₂<82> is, in particular, the low-pressure vapour from the condensate vessel <86>. The two are mixed via the regulating unit <90> and conveyed via the outlet as mixed steam H₄<84> to the appropriate heat exchanger WT, e.g. <W8>, <W9>, <W13>. The amount of driving vapour and suction vapour can be set via the regulating unit <90>, so that pressure and thus the condensation temperature of the mixed steam H₄<84> can be set.

### 4. DETAILED DESCRIPTION OF THE INVENTION

### 4.1 Step (a1) of the Process According to the Invention

In step (a1) of the process according to the invention, a feed stream S_{AE1} comprising ROH is reacted with a feed stream S_{AE2} comprising M_{A}OH in countercurrent in a reactive rectification column RR_{A} to afford a crude product mixture RP_{A} comprising M_{A}OR, water, ROH, M_{A}OH.

According to the invention, a “reactive rectification column” is a rectification column in which the reaction according to step (a1) or step (a2) of the process of the invention proceeds at least in some parts. It may also be abbreviated to “reaction column”.

In step (a1) of the process according to the invention, a bottom product stream S_{AS} comprising ROH and M_{A}OR is withdrawn at the lower end of RR_{A}. A vapour stream S_{AB} comprising water and ROH is withdrawn at the upper end of RR_{A}.

“Vapour stream” means that the respective stream is a gaseous stream.

In the process according to the invention, R is a C₁-C₆-hydrocarbon radical, preferably selected from the group consisting of methyl, ethyl, n-propyl, isopropyl, n-butyl, sec-butyl, isobutyl, tert-butyl, isomers of pentyl such as n-pentyl, more preferably selected from the group consisting of methyl, ethyl, n-propyl, isopropyl, yet more preferably selected from the group consisting of methyl and ethyl. R is particularly preferably methyl and ROH is accordingly methanol.

M_{A} is selected from sodium, potassium, preferably sodium.

The feed stream S_{AE1} comprises ROH. In a preferred embodiment, the proportion by mass of ROH in S_{AE1} is, based on the total mass of the feed stream S_{AE1}, ≥95% by weight, even more preferably 99% by weight, where S_{AE1} additionally comprises, in particular, water.

The alcohol ROH used as feed stream S_{AE1} in step (a1) of the process of the invention can also be a commercial alcohol having a proportion by mass of alcohol, based on the total mass of the feed stream S_{AE1}, of more than 99.8% by weight and a proportion by mass of water, based on the total mass of the feed stream S_{AE1}, of up to 0.2% by weight.

The feed stream S_{AE1} is preferably introduced in vapour form.

The feed stream S_{AE2} comprises M_{A}OH, preferably in a proportion by mass of from 10 to 75% by weight, more preferably from 20 to 55% by weight, more preferably 48.5% by weight, in each case based on the total mass of the stream S_{AE2}. In a preferred embodiment, S_{AE2} comprises not only M_{A}OH but also at least one further compound selected from water, ROH. S_{AE2} more preferably comprises water in addition to M_{A}OH, in which case S_{AE2} is then an aqueous solution of M_{A}OH.

Step (a1) of the process of the invention is carried out in a reactive rectification column RR_{A}. Step (a2) of the process of the invention is carried out in a reactive rectification column RR_{B}.

The reaction column RR_{A} or RR_{B} preferably contains internals. Suitable internals are, for example, trays, structured packings or unstructured packings. When the reaction column RR_{A} or RR_{B} contains trays, then bubble cap trays, valve trays, tunnel trays, Thormann trays, cross-slit bubble cap trays or sieve trays are suitable. When the reaction column RR_{A} or RR_{B} contains trays, then preference is given to choosing trays in the case of which not more than 5% by weight, more preferably less than 1% by weight, of the liquid trickles through the respective trays. The constructional measures required to minimize trickle-through of the liquid are familiar to those skilled in the art. In the case of valve trays, particularly tightly closing valve designs are selected for example. Reducing the number of valves also makes it possible to increase the vapour velocity in the tray openings to twice the value typically established. When sieve trays are used, it is particularly advantageous to reduce the diameter of the tray openings and maintain the number of openings.

When using structured or unstructured packings, structured packings are preferred in terms of uniform distribution of the liquid.

For columns comprising unstructured packings, especially comprising random packings, and for columns comprising structured packings, the desired characteristics of the liquid distribution may be achieved when the liquid trickling density in the edge region of the column cross section adjacent to the column shell, corresponding to about 2% to 5% of the total column cross section, is reduced compared to the other cross-sectional regions by up to 100%, preferably by 5% to 15%. This can easily be achieved by, for example, targeted distributions of the drip points of the liquid distributors or the holes thereof.

In order to avoid droplet entrainment, the columns can additionally comprise appropriate internals known to those skilled in the art, for example knitted wire droplet precipitators. Examples of such droplet precipitators, for example those marketed under the name “KnitMesh V-MISTER™-Technology” by Sulzer, are described in the brochure “Gas/Liquid Separation Technology” E10508 en 4.2018, Copyright © Sulzer Ltd 2018, which can be found online at: https://www.sulzer.com/-/media/files/products/separation-technology/feed-inlet-devices/gas_liquid_separation_technology.ashx?la=en.

The process according to the invention may be carried out either continuously or discontinuously. It is preferably carried out continuously.

According to the invention, “Reaction of a feed stream S_{AE1} comprising ROH with a feed stream S_{AE2} comprising M_{A}OH in countercurrent” is achieved, in particular, as a result of the feed point for at least part of the feed stream S_{AE1} comprising ROH in step (a1) being located on the reaction column RR_{A} below the feed point of the feed stream S_{AE2} comprising M_{A}OH.

The reaction column RR_{A} preferably comprises at least 2, in particular 15 to 40, theoretical plates between the feed point of the feed stream S_{AE1} and the feed point of the feed stream S_{AE2}.

In particular, the feed stream S_{AE1} comprising ROH is introduced in vapour form in the lower region of the reaction column RR_{A}. Step (a1) of the process according to the invention also encompasses the case where a portion of the feed stream S_{AE1} comprising ROH is added in vapour form below the feed point of the feed stream S_{AE2} comprising aqueous sodium hydroxide solution M_{A}OH but nevertheless at the upper end or in the region of the upper end of the reaction column RR_{A}. This makes it possible to reduce the dimensions of the lower region of the reaction column RR_{A}. When part of the feed stream S_{AE1} comprising ROH, in particular methanol, is introduced, in particular in vapour form, at the upper end or in the region of the upper end of the reaction column RR_{A}, then only a partial amount of from 10 to 70% by weight, preferably from 30 to 50% by weight [in each case based on the total amount of the alcohol ROH used in step (a1)] is fed in at the lower end of the reaction column RR_{A} and the remaining partial amount is introduced in vapour form as a single stream or distributed over a plurality of substreams, preferably from 1 to 10 theoretical plates, particularly preferably from 1 to 3 theoretical plates, below the point of addition of the feed stream S_{AE2} comprising M_{A}OH.

In the reaction column RR_{A}, the feed stream S_{AE1} comprising ROH is then reacted with the feed stream S_{AE2} comprising M_{A}OH according to the reaction <1> described hereinabove to afford M_{A}OR and H₂O, where these products are present in admixture with the reactants ROH and M_{A}OH since an equilibrium reaction is concerned. Accordingly, a crude product mixture RP_{A} which comprises ROH and M_{A}OH in addition to the products M_{A}OR and water is obtained in step (a1) of the process of the invention in the reaction column RR_{A}.

At the lower end of RR_{A}, the bottom product stream S_{AS} (“S_{AS}” can also be referred to as “S_{AP}”) comprising ROH and M_{A}OR is then taken off.

At the upper end of RR_{A}, the alcohol stream which still contains water, referred to above as “vapor stream S_{AB} comprising water and ROH”, is then taken off.

The amount of the alcohol ROH present in the feed stream S_{AE1} is preferably selected so that it simultaneously serves as solvent for the alkali metal alkoxide M_{A}OR present in the bottom product stream S_{AS}.

In a preferred embodiment of the process of the invention, especially in cases in which S_{AE2} comprises water in addition to M_{A}OH, the ratio of the total weight (masses; unit: kg) of alcohol ROH used as feed stream S_{AE1} in step (a1) to the total weight (masses; unit: kg) of M_{A}OH used as feed stream S_{AE2} in step (a1) is from 2:1 to 50:1, more preferably 5:1 to 48:1, yet more preferably 10:1 to 35:1, yet still more preferably 13:1 to 30:1.

The reaction column RR_{A} is operated with or without, preferably without, reflux.

“Without reflux” means that the vapor stream S_{AB} comprising water and ROH taken off at the upper end of RR_{A} or the vapour stream S_{BB} comprising water and ROH taken off in the optional step (a2) at the upper end of RR_{B} is fed in its entirety to the rectification column RD_{A} according to step (b). The vapour stream S_{AB} or the vapor stream S_{AB} taken off in the optional step (a2) is preferably fed in vapor form to the first rectification column RD_{A}.

“With reflux” means that the vapor stream S_{AB} which comprises water and ROH and is taken off at the upper end of the respective column, in step (a1) the reaction column RR_{A}, and the vapour stream S_{BB} which comprises water and ROH and is taken off in the optional step (a2) from the reaction column RR_{B} is not discharged in its entirety from RR_{A} or RR_{B}, i.e. is not introduced in its entirety into the rectification column RD_{A} but is instead partly fed back as runback to the respective column, in step (a1) S_{AB}, i.e. into the reaction column RR_{A}, and in the optional step (a2) S_{BB}, i.e. the reaction column RR_{B}. In the cases where such a reflux is established, the reflux ratio is preferably 0.005 to 0.99, more preferably 0.01 to 0.9, yet more preferably 0.02 to 0.34, particularly preferably 0.025 to 0.27 and very particularly preferably 0.03 to 0.24. Reflux can be established by a condenser K_{RRA} being installed at the top of the respective column, in step (a1) the reaction column RR_{A} and in the optional step (a2) a condenser K_{RRB} being installed on the reaction column RR_{B}, in which condenser the vapour stream S_{AB} or S_{BB} is at least partially condensed and fed back to the respective column RR_{A} or RR_{B}. Generally and in the context of the present invention, a reflux ratio is to be understood as meaning the ratio of the mass flow (kg/h) recycled to the respective column in liquid form (reflux or runback) to the mass flow (kg/h) discharged from the respective column in liquid form (distillate) or gaseous form (vapour).

In the embodiment in which a reflux is established on the reaction column RR_{A}, the alkali metal hydroxide M_{A}OH employed in step (a1) as feed stream S_{AE2} may also be at least partially mixed with the reflux stream and the resulting mixture thus supplied to step (a1).

Step (a1) of the process of the invention is, in particular, carried out at a temperature T_{RRA} in the range from 45° C. to 190° C., preferably from 47° C. to 170° C., more preferably from 60° C. to 150° C., and in particular at a pressure p_{RRA} of from 0.5 bar to 40 bar, preferably in the range from 0.75 bar to 20 bar, more preferably in the range from 0.9 bar to 10 bar, more preferably in the range from 1 bar to 7 bar, even more preferably from 1 bar to 5 bar.

The reaction column RR_{A} comprises, in a preferred embodiment, at least one vaporizer which is, in particular, selected from intermediate vaporizers VZ_{RRA} and bottom vaporizers VS_{RRA}, even more preferably at least one bottom vaporizer VS_{RRA}.

For the purposes of the invention, the expression “intermediate vaporizer” VZ_{RRA} or VZ_{RRB} refers to vaporizers which are located above the liquid phase of the respective column, in particular above the liquid phase of the reaction column RR_{A} or RR_{B}. They are used, in particular, to vaporize crude product mixture RP_{A} or RP_{B}.

For the purposes of the invention, the expression “bottom vaporizer” VS_{RRA} and VS_{RRB} refers to vaporizers which heat the liquid phase of the respective column, in particular the liquid phase of the reaction column RR_{A} or RR_{B}. In these, bottom product stream S_{AS} or S_{BS} (“S_{BS}” can also be referred to as “S_{BP}”) is at least partially vaporized.

A vaporizer is typically arranged outside the respective reaction column or rectification column. The mixture to be vaporized in the vaporizer is taken off from the column via an offtake or “offtake point” and fed to the at least one vaporizer.

In the case of intermediate vaporization of the crude product mixture RP_{A} or RP_{B} on the reaction column RR_{A} or RR_{B}, this mixture is taken off as stream S_{RRAZ} or S_{RRBZ} and fed to the at least one intermediate vaporizer VZ_{RRA} or VZ_{RRB}. The vaporized mixture, optionally with a residual proportion of liquid, is recirculated via an inlet or “addition point” back into the respective column RR_{A} or RR_{B}. When the vaporizer is an intermediate vaporizer, i.e., in particular, is an intermediate vaporizer VZ_{RRA} or VZ_{RRB}, the offtake via which the respective mixture is taken off and fed to the vaporizer is a side offtake and the inlet via which the respective vaporized mixture is returned to the column is a side inlet. When the vaporizer is a bottom vaporizer, i.e. heats the liquid phase or bottoms in the column, it is, in particular, a bottom vaporizer VS_{RRA} or VS_{RRB}, so that at least part of the bottom offtake stream, in particular S_{AS} or S_{BS}, is fed to the corresponding bottom vaporizer, vaporized and fed back into the respective column in the region of the bottom.

However, it is also possible as an alternative to provide tubes through which the appropriate heating medium, for example H₁ or H₄ flows, for example on a suitable tray when using an intermediate vaporizer or in the bottom region of the respective column. In this case, the vaporization occurs on the tray or in the bottom region of the column. However, it is preferable to arrange the vaporizer outside the respective column.

Suitable vaporizers VD which can be used as intermediate vaporizers VZ and bottom vaporizers VS are, for example, natural convection vaporizers, forced circulation vaporizers, forced circulation vaporizers with depressurization, tank vaporizers, falling-film evaporators or thin-film evaporators. A shell-and-tube arrangement or plate apparatus is usually employed as heat-transferring component for the vaporizer in the case of natural convection vaporizers and forced circulation vaporizers. When using a shell-and-tube heat exchanger, the heat transfer medium, for example H₁ or H₄, can either flow through the tubes and the mixture to be vaporized flows around the tubes or else the heat transfer medium flows around the tubes and the mixture to be vaporized flows through the tubes. In the case of a falling-film evaporator, the mixture to be vaporized is typically introduced as a thin film on the inside of a tube and the tube is heated externally. In contrast to a falling-film evaporator, a thin-film evaporator additionally comprises a rotor with wipers which distributes the liquid to be evaporated on the inner wall of the tube to form a thin film.

In addition to the abovementioned types of vaporizer, it is also possible to use any other type of vaporizer which is known to a person skilled in the art and is suitable for use in a rectification column or reaction column.

When the reaction column RR_{A} or reaction column RR_{B} comprises an intermediate vaporizer VZ_{RRA} or VZ_{RRB}, preference is given to the respective intermediate vaporizer being arranged in the region of the feed point of the feed stream S_{AE1} in the case of the reaction column RR_{A} or in the region of the feed point of the feed stream S_{BE1} in the case of the reaction column RR_{B}. In this way, a predominant part of the heat energy can be introduced via the intermediate vaporizer VZ_{RRA} or VZ_{RRB}. It is thus possible for example to introduce more than 80% of the energy via the intermediate vaporizer. According to the invention, the intermediate vaporizer VZ_{RRA} or VZ_{RRB} is preferably arranged and/or configured so that more than 50%, in particular more than 75%, of the total energy required for the reactive rectification can be introduced by means of it.

When the reaction column RR_{A} or reaction column RR_{B} comprises an intermediate vaporizer VZ_{RRA} or VZ_{RRB}, it is also advantageous for the intermediate vaporizer to be arranged so that the reaction column RR_{A} or RR_{B} has from 1 to 50, preferably from 1 to 40, theoretical plates below the intermediate vaporizer and has from 1 to 200, preferably from 1 to 40, theoretical plates above the intermediate vaporizer. In particular, the reaction column RR_{A} or RR_{B} then preferably has from 2 to 10 theoretical plates below the intermediate vaporizer and has from 20 to 50 theoretical plates above the intermediate vaporizer.

When the reaction column RR_{A} or reaction column RR_{B} comprises an intermediate vaporizer VZ_{RRA} or VZ_{RRB}, it is also advantageous for the side offtake (i.e. the “offtake point E_{RRA}” on the reaction column RR_{A} or the “offtake point E_{RRB}” on the reaction column RR_{B}) via which the crude product mixture RP_{A} or RP_{B} is fed to the intermediate vaporizer VZ_{RRA} or VZ_{RRB} and the side inlet (i.e. the “feed point Z_{RRA}” on the reaction column RR_{A} or the “feed point Z_{RRB}” on the reaction column RR_{B}) via which the vaporized crude product mixture RP_{A} or RP_{B} from the intermediate vaporizer VZ_{RRA} or VZ_{RRB} is fed back into the respective reaction column RR_{A} or RR_{B} to be not more than two theoretical plates from one another, preferably positioned between the same plates of the reaction column RR_{A} or reaction column RR_{B}. However, it is also possible for the side offtake and side inlet to be at different heights.

It is also advantageous, when the reaction column RR_{A} or the reaction column RR_{B} comprises an intermediate vaporizer VZ_{RRA} or VZ_{RRB}, for the “offtake point E_{RRA}” on the reaction column RR_{A} or the “offtake point E_{R}RB” on the reaction column RR_{B} and the “feed point Z_{RRA}” on the reaction column RR_{A} or the “feed point Z_{RRB}” on the reaction column RR_{B} to be located below the feed points of S_{AE2} or S_{BE2}.

In a preferred embodiment when using an intermediate vaporizer VZ_{RRA} or VZ_{RRB} in RR_{A} or RR_{B}, the diameter of the reaction column RR_{A} or RR_{B} above the intermediate vaporizer RR_{A} or RR_{B} is greater than the diameter of the reaction column RR_{A} or RR_{B} below the intermediate vaporizer VZ_{RRA} or VZ_{RRB}. This has the advantage of allowing capital expenditure savings.

In such an intermediate vaporizer VZ_{RRA} or VZ_{RRB}, the liquid crude product mixture RP_{A} comprising M_{A}OR, water, ROH, M_{A}OH present in the reaction column RR_{A} or liquid crude product mixture RP_{B} comprising M_{B}OR, water, ROH, M_{B}OH present in the reaction column RR_{B} can be brought into the gaseous state.

As a result of the arrangement of one or more intermediate vaporizers VZ_{RRA} in the upper region of the reaction column RR_{A} or of one or more intermediate vaporizers VZ_{RRB} in the upper region of the reaction column RR_{B}, the dimensions in the lower region of the reaction column RR_{A} or RR_{B} can be reduced. In the embodiment having at least one intermediate vaporizer, preferably a plurality of intermediate vaporizers VZ_{RRA} or VZ_{RRB}, it is also possible to introduce substreams of the ROH in liquid form in the upper region of the reaction column RR_{A} or RR_{B}.

In step (a1) of the process of the invention, a bottom product stream S_{AS} comprising ROH and M_{A}OR is taken off at the lower end of the reaction column RR_{A}.

Preference is given to the reaction column RR_{A} having at least one bottom vaporizer VS_{RRA} via which the bottom product stream S_{AS} is then at least partly conveyed.

Bottom vaporizers are, according to the invention, arranged at the bottom of the reaction column RR_{A} or RR_{B} and are then referred to as “VS_{RRA}” or “VS_{RRB}”. In such a bottom vaporizer, bottom product stream S_{AS} or S_{BS} present in the reaction column RR_{A} or RR_{B} can be conveyed and ROH can be at least partly removed therefrom, as a result of which the proportion by mass of M_{A}OR or M_{B}OR in the respective bottom stream can be set in a targeted manner.

The bottom product stream S_{AS} taken off in step (a1) preferably has a proportion by mass of M_{A}OR in ROH in the range from 1 to 50% by weight, preferably from 5 to 32% by weight, more preferably from 10 to 32% by weight, even more preferably from 15 to 32% by weight, in each case based on the total mass of S_{AS}.

The proportion by mass of residual water in S_{AS} is preferably <1% by weight, preferably <0.5% by weight, more preferably <0.2% by weight, based on the total mass of S_{AS}.

The proportion by mass of starting material M_{A}OH in S_{AS} is preferably <1% by weight, preferably <0.5% by weight, more preferably <0.2% by weight, based on the total mass of S_{AS}.

### 4.2 Step (a2) of the Process According to the Invention (Optional)

According to the invention step (a2) is optionally performed. In the optional step (a2), which proceeds simultaneously with and spatially separately from step (a1) of the process of the invention, a feed stream S_{BE1} comprising ROH is reacted with a feed stream S_{BE2} comprising M_{B}OH in countercurrent in a reactive rectification column RR_{B} to give a crude product mixture RP_{B} comprising M_{B}OR, water, ROH, M_{B}OH.

In the optional step (a2) of the process of the invention, a bottom product stream S_{BS} comprising ROH and M_{B}OR is taken off at the lower end of RR_{B}. A vapour stream S_{BB} comprising water and ROH is withdrawn at the top end of RR_{B}.

M_{B} is selected from sodium, potassium, preferably potassium.

The feed stream S_{BE1} comprises ROH. In a preferred embodiment, the proportion by mass of ROH in S_{BE1} is, based on the total mass of the feed stream S_{BE1}, ≥95% by weight, even more preferably 99% by weight, with S_{BE1} otherwise comprising, in particular, water.

The alcohol ROH used as feed stream S_{BE1} in the optional step (a2) of the process of the invention can also be a commercial alcohol having a proportion by mass of alcohol, based on the total mass of the feed stream S_{BE1}, of more than 99.8% by weight and a proportion by mass of water, based on the total mass of the feed stream S_{BE1}, of up to 0.2% by weight.

The feed stream S_{BE1} is preferably introduced in vapour form.

The feed stream S_{BE2} preferably comprises M_{B}OH in a proportion by mass of from 10 to 75% by weight, more preferably from 20 to 55% by weight, more preferably from 48 to 52% by weight, in each case based on the total mass of the stream S_{BE2}. In a preferred embodiment S_{BE2} comprises not only M_{B}OH but also at least one further compound selected from water, ROH. It is yet more preferable when S_{BE2} comprises water in addition to M_{B}OH, thus rendering S_{BE2} an aqueous solution of M_{B}OH.

Step (a2) of the process according to the invention is performed in a reactive rectification column (or “reaction column”) RR_{B}. Preferred embodiments of the reaction column RR_{B} are described in section 4.1.

According to the invention “reaction of a feed stream S_{BE1} comprising ROH with a feed stream S_{BE2} comprising M_{B}OH in countercurrent” is especially achieved as a result of the feed point for at least a portion of the feed stream S_{BE1} comprising ROH in step (a2) being arranged below the feed point for the feed stream S_{BE2} comprising M_{B}OH on the reaction column RR_{B}.

The reaction column RR_{B} preferably comprises at least 2, in particular 15 to 40, theoretical plates between the feed point of the feed stream S_{BE1} and the feed point of the feed stream S_{BE2}.

In particular, the feed stream S_{BE1} comprising ROH is introduced in vapour form in the lower region of the reaction column RR_{B}. The optional step (a2) of the process of the invention also encompasses the case of part of the feed stream S_{BE1} comprising ROH being introduced in vapour form below the feed point of the feed stream S_{BE2} comprising aqueous sodium hydroxide solution M_{B}OH but at the upper end or in the region of the upper end of the reaction column RR_{B}. This makes it possible to reduce the dimensions of the lower region of the reaction column RR_{B}. When part of the feed stream S_{BE1} comprising ROH, in particular methanol, is introduced, in particular in vapour form, at the upper end or in the region of the upper end of the reaction column RR_{B}, then only a partial amount of from 10 to 70% by weight, preferably from 30 to 50% by weight [in each case based on the total amount of the alcohol ROH used in the optional step (a2)] is fed in at the lower end of the reaction column RR_{B} and the remaining partial amount is introduced in vapour form in a single stream or distributed over a plurality of substreams, preferably from 1 to 10 theoretical plates, particularly preferably from 1 to 3 theoretical plates, below the feed point of the feed stream S_{BE2} comprising M_{B}OH.

In the reaction column RR_{B} the feed stream S_{BE1} comprising ROH is then reacted with the feed stream S_{BE2} comprising M_{B}OH according to the reaction <1> described hereinabove to afford M_{B}OR and H₂O, where these products are present in admixture with the reactants ROH and M_{B}OH since an equilibrium reaction is concerned. Accordingly, a crude product mixture RP_{B} which comprises the products M_{B}OR and water together with ROH and M_{B}OH is obtained in the optional step (a2) of the process of the invention in the reaction column RR_{B}.

The bottom product stream S_{BS} (“S_{BS}” can also be referred to as “S_{BP}”) comprising ROH and M_{B}OR is then obtained and taken off at the lower end of RR_{B}.

The alcohol stream additionally containing water, hereinafter referred to as “vapour stream S_{BB} comprising water and ROH” is then taken off at the upper end of RR_{B}.

The amount of alcohol ROH present in the feed stream S_{BE1} is preferably selected so that it simultaneously serves as solvent for the alkali metal alkoxide M_{B}OR present in the bottom product stream S_{BP}.

In a preferred embodiment of the optional step (a2) of the process of the invention, and especially in cases in which S_{BE2} comprises water in addition to M_{B}OH, the ratio of the total weight (masses; unit: kg) of alcohol ROH used as feed stream S_{BE1} in step (a2) to the total weight (masses; unit: kg) of M_{B}OH used as feed stream S_{BE2} in step (a2) is from 2:1 to 50:1, more preferably 5:1 to 48:1, yet more preferably 10:1 to 35:1, yet still more preferably 13:1 to 30:1.

This vapour stream S_{BB} comprising water and ROH is supplied to step (b) of the process according to the invention. It is mixed with S_{AB} before being supplied to step (b) of the process of the invention or supplied separately from S_{AB} to step (b) of the process of the invention. Vapour stream S_{BB} is preferably mixed with S_{AB} and the resulting mixed vapour stream is then introduced into step (b) of the process of the invention.

The reaction column RR_{B} is operated with or without, preferably without, reflux.

In the embodiment in which reflux is established on the reaction column RR_{B}, the alkali metal hydroxide M_{B}OH used as feed stream S_{BE2} in the optional step (a2) can also be mixed at least partly with the reflux stream and the resulting mixture can thus be supplied to the optional step (a2).

The optional step (a2) of the process of the invention is, in particular, carried out at a temperature T_{R}RB in the range from 45° C. to 190° C., preferably from 47° C. to 170° C., more preferably from 60° C. to 150° C., and in particular at a pressure p_{RRB} of from 0.5 bar to 40 bar, preferably in the range from 0.75 bar to 20 bar, more preferably in the range from 0.9 bar to 10 bar, more preferably in the range from 1 bar to 7 bar, even more preferably from 1 bar to 5 bar.

In a preferred embodiment, the reaction column RR_{B} comprises at least one vaporizer which is, in particular, selected from intermediate vaporizers VZ_{RRB} and bottom vaporizers VS_{RRB}. The reaction column RR_{B} particularly preferably comprises at least one bottom vaporizer VS_{RRB}.

In the optional step (a2) of the process of the invention, a bottom product stream S_{B}S comprising ROH and M_{B}OR is taken off at the lower end of the reaction column RR_{B}.

Preference is given to the reaction column RR_{B} comprising at least one bottom vaporizer VS_{RRB} via which the bottom product stream S_{BS} is then at least partly conveyed.

The bottom product stream S_{B}S taken off in step (a2) preferably has a proportion by mass of M_{B}OR in ROH in the range from 1 to 50% by weight, preferably from 5 to 32% by weight, more preferably from 10 to 32% by weight, even more preferably from 15 to 32% by weight, in each case based on the total mass of S_{BS}.

The proportion by mass of residual water in S_{BS} is preferably <1% by weight, preferably <0.5% by weight, more preferably <0.2% by weight, based on the total mass of S_{BS}.

The proportion by mass of starting material M_{B}OH in S_{BS} is preferably <1% by weight, preferably <0.5% by weight, more preferably <0.2% by weight, based on the total mass of S_{BS}.

In a preferred embodiment of the present process, step (a2) is carried out.

This has the advantage that two different alkali metal alkoxides can be produced without causing cross-contamination of the one alkali metal alkoxide with the other or product losses occurring on changing over of the starting material M_{A}OH to M_{B}OH.

The optional step (a2) of the process of the invention is carried out simultaneously with and spatially separately from step (a1). Spatial separation is ensured by performing steps (a1) and (a2) in the two reaction columns RR_{A} and RR_{B}.

In an advantageous embodiment of the invention the reaction columns RR_{A} and RR_{B} are accommodated in one column shell, where the column is at least partially subdivided by at least one dividing wall. Such a column having at least one dividing wall will according to the invention be referred to as “DWC”. Such dividing wall columns are familiar to those skilled in the art and are described for example in U.S. Pat. No. 2,295,256, EP 0 122 367 A2, EP 0 126 288 A2, WO 2010/097318 A1 and I. Dejanović, Lj. Matijašević, Ž. Olujić, Chemical Engineering and Processing 2010, 49, 559-580. In the dividing wall columns suitable for the process according to the invention the dividing walls preferably extend to the column floor and, in particular, preferably span at least a quarter, more preferably at least a third, yet more preferably at least half, yet more preferably at least two thirds, yet still more preferably at least three quarters, of the column by height They divide the columns into at least two reaction spaces in which spatially separate reactions may be carried out. The reaction spaces provided by the at least one dividing wall may be of identical or different sizes.

In the regions separated by the dividing wall, the bottom product streams S_{AS} and S_{BS} can, in this embodiment, be taken off separately and preferably be conveyed via the bottom vaporizer VS_{RRA} or VS_{RRB} installed for each reaction space formed by the at least one reaction wall, in which a desired concentration of M_{A}OR or M_{B}OR can be set by removal of ROH from S_{AS} or S_{BS}.

### 4.3 Step (b) of the Process According to the Invention

In step (b) of the process of the invention, the vapour stream S_{AB} and, when step (a2) is carried out, the vapour stream S_{BB}, mixed with S_{AB} or separately from S_{AB} is introduced into a rectification column RD_{A} so that a mixture G_{A} comprising water and ROH is present in the rectification column RD_{A}.

In the optional form of the process of the invention in which step (a2) is carried out, the vapour stream S_{BB} is preferably mixed with S_{AB} and the mixed vapour obtained is then introduced into the rectification column RD_{A}.

In one embodiment of the present invention, the vapour stream S_{AB} and, in cases in which the optional step (a2) is carried out, the vapour stream S_{BB} can be compressed before they are introduced into the rectification column RD_{A}. This can occur by means of a compressor VD₃₁.

A person skilled in the art can provide one or more compressors between the columns on the basis of such a person's specialist knowledge. Particularly in the case of gaseous streams which connect two or more columns, a compression/compression step should always be provided when the pressure in the column into which a gaseous stream is fed is greater than the pressure in the column from which the stream concerned exits.

It goes without saying that even in the embodiments in which the optional step (a2) is carried out and S_{BB} is introduced separately from S_{AB} into the rectification column RD_{A}, S_{AB} and S_{BB} mix in the rectification column RD_{A}, so that a mixture G_{A} comprising water and ROH is in every case obtained in the rectification column RD_{A} after step (b) has been carried out.

As rectification column RD_{A} in step (b) of the process of the invention, it is possible to use rectification columns known to a person skilled in the art. The rectification column RD_{A} preferably contains internals. Suitable internals are, for example, trays, unstructured packings or structured packings. As trays, use is normally made of bubble cap trays, sieve trays, valve trays, tunnel trays or slit trays. Unstructured packings are generally beds of random packing elements. Packing elements normally used are Raschig rings, Pall rings, Berl saddles or Intalox® saddles. Structured packings are for example marketed under the trade name Mellapack® from Sulzer. Apart from the internals mentioned, further suitable internals are known to a person skilled in the art and can likewise be used.

Preferred internals have a low specific pressure drop per theoretical plate. Structured packings and random packing elements have, for example, a significantly lower pressure drop per theoretical plate than trays. This has the advantage that the pressure drop in the rectification column remains very low and the mechanical load on the compressor and the temperature of the alcohol/water mixture G_{A} to be vaporized remain low.

When the rectification column RD_{A} contains structured packings or unstructured packings these may be divided or in the form of an uninterrupted packing. However, it is usual to provide at least two packing sections, one packing section above the feed point for the vapour stream to be separated (this is S_{AB} or, mixed or separately from one another, S_{AB} and S_{BB}) and a packing section below the feed point for the vapour stream to be separated. When a packing is used, for example a bed of random packing elements, the packing elements usually rest on a suitable support system. Preference is given to an embodiment in which packings are installed in the rectification column RD_{A} above the inlet and trays are installed below the inlet.

The rectification column RD_{A} can also comprise internals for preventing entrainment of droplets as have been described for the reaction column RR_{A}.

In an advantageous embodiment of the invention, the reaction column RR_{A} of step (a1), or reaction columns RR_{A} and RR_{B} in the above-described preferred embodiment in which step (a2) is carried out, and the rectification column RD_{A} of step (b) for separation of the mixture G_{A} are accommodated in a column shell, with the column being divided at least partly by at least one dividing wall, or in the above-described preferred embodiment in which step (a2) is carried out, by at least two dividing walls, with the at least one dividing wall or the at least two dividing walls extending to the bottom of the column. As described at point 4.2 this is then a dividing wall column again.

In this case, the reaction to form the crude product mixture RP_{A} as per step (a1) or the crude product mixtures RP_{A} and RP_{B} as per steps (a1) and (a2) is carried out in part of the DWC, with the feed stream S_{AE2} and optionally the feed stream S_{BE2} being introduced below but at approximately the height of the upper end of the dividing wall and the feed stream S_{AE1} and optionally the feed stream S_{BE1} being introduced in vapour form at the lower end. The alcohol/water mixture formed above the feed point of the feed stream then becomes distributed above the dividing wall over the entire column region which serves as enrichment section of the rectification portion RD_{A}. The second, lower part of the column which has been separated off by the dividing wall is the stripping section of the rectification column RD_{A}. The energy required for the distillation is then supplied via a vaporizer at the lower end of the second part of the column separated off by the dividing wall, with this vaporizer being able to be heated conventionally or be heated by means of heating vapour, for example H₁ or H₄. When the vaporizer is heated conventionally, an intermediate vaporizer which can be heated by means of heating vapour, for example H₁ or H₄, can be additionally provided.

At the end of step (b) of the process of the invention, a mixture G_{A} comprising water and ROH is finally obtained in the rectification column RD_{A}. The composition of the mixture G_{A} is determined, in particular, by the composition of the vapour stream S_{AB} or, when step (a2) is carried out, in particular proportionately by the composition of the two vapour streams S_{AB} and S_{BB}.

### 4.4 Step (c) of the Process According to the Invention

In step (c) of the process of the invention, the mixture G_{A} comprising water and ROH is separated in RD_{A} into an ROH-comprising vapour stream S_{DAB} at the upper end of RD_{A} and a bottom stream S_{DAS} comprising water and ROH at the lower end of RD_{A}.

The pressure p_{A} in RD_{A} can be set by a person skilled in the art on the basis of the person's specialist knowledge. It is preferably in the range from 1 bar to 20 bar, preferably from 1 bar to 15 bar, more preferably from 1 to 10 bar.

The temperature T_{A} in RD_{A} can be set by a person skilled in the art on the basis of such a person's specialist knowledge. It is preferably in the range from 40° C. to 220° C., preferably from 60° C. to 190° C.

The separation according to step (c) of the process of the invention is a fractional distillation of the alcohol/water mixture G_{A} as is known to a person skilled in the art.

At the bottom (another word for “bottom” is “at the lower end”) of the rectification column RD_{A}, a stream S_{DAS} comprising alcohol is obtained.

At the top (another word for “top” is “at the upper end”) of the rectification column RD_{A}, the vapour stream S_{DAB} comprising ROH is additionally obtained. The preferred proportion by mass of ROH in this vapour stream S_{DAB} is ≥99% by weight, preferably ≥99.6% by weight, more preferably 99.9% by weight, in each case based on the total mass of S_{DAB}, with the balance being, in particular, water.

The bottom stream S_{DAS} is discharged from RD_{A} at an offtake point E_{AS} at the lower end of RD_{A} and the vapour stream S_{DAB} is discharged from RD_{A} at an offtake point E_{AK} at the upper end of RD_{A}.

For the purposes of the present invention, “offtake point E_{AK} at the upper end of RD_{A}” means that E_{AK} is installed on the column in such a way that the vapour stream S_{DAB} is taken off as overhead stream or as side offtake stream above the internals in the rectification column RD_{A}.

For the purposes of the present invention, “offtake point E_{AS} at the lower end of RD_{A}” means that E_{AS} is installed on the column in such a way that the bottom stream S_{DAS} is taken off as bottom stream or as side offtake stream below the internals in the rectification column RD_{A}.

In an optional embodiment of the process of the invention, at least one further stream S_{AZ} which comprises ROH and water and is different from S_{DAS} and S_{DAB} is discharged from RD_{A}, energy is transferred to this stream and the stream is recirculated into RD_{A}, where the position of the offtake point E_{AZ} on RD_{A} is located between the two offtake points E_{AS} and E_{AK}. The stream S_{AZ} comprises the mixture G_{A}.

In this optional embodiment of step (c) of the process of the invention, it is possible to supply energy to the mixture G_{A}. In a preferred embodiment, this occurs by the mixture G_{A} being taken off as stream S_{AZ} and conveyed via an intermediate vaporizer VZ_{A} in which energy is transferred from a heat transfer medium, in particular heating vapour H₁ or H₄ or a different heating medium W₁ to S_{AZ} or G_{A}. This energy transfer can advantageously be carried out by the mixture G_{A} being conveyed as stream S_{AZ} and W₁ or the mixture G_{A} being conveyed as stream S_{AZ} and the heating vapour H₁ or H₄ being conveyed through an intermediate vaporizer VZ_{A}.

At least part of the alcohol ROH obtained in the vapour stream S_{DAB} in the distillation is preferably fed to the reaction column RR_{A} at least as part of the feed stream S_{AE1} and, in the cases in which step (a2) is carried out in the process of the invention, alternately or additionally to the reaction column RR_{B} at least as part of the feed stream S_{BE1}.

In step (c), the vapour S_{AB} or S_{AB} and S_{BB} obtained in step (a1) or step (a1) and (a2) is separated by distillation. These vapours comprise essentially the alcohol ROH and water. In particular, S_{AB} or S_{AB} and S_{BB} are each a water/alcohol mixture in which the proportion by mass of ROH is preferably in the range >80% by weight, more preferably >85% by weight, even more preferably >90% by weight (based on the total mass of S_{AB} or S_{AB} and S_{BB}). Thus, G_{A} is in particular an alcohol/water mixture in which the proportion by mass of ROH is preferably in the range >80% by weight, more preferably >95% by weight, even more preferably >90% by weight (based on the total mass of G_{A}).

The bottom stream S_{DAS} taken off in step (c) comprises ROH in a proportion by mass of, in particular, from 0.005 to 95% by weight, based on the total mass of S_{DAS}. Preference is given to from 25 to 95% by weight in the cases in which step (d) is carried out and from 0.005 to 3% by weight in the cases in which step (d) is not carried out, in each case based on the total mass of S_{DAS}. S_{DAS} preferably comprises essentially water in addition to the alcohol ROH.

The alcohol ROH is consumed in the process according to the invention and especially in a continuous process mode therefore requires replacement with fresh alcohol ROH.

Fresh alcohol ROH is, in particular, introduced into at least one of the columns selected from reactive rectification column RD_{A}, reaction column RR_{A} and, when step (a2) is carried out, alternatively or additionally into the reaction column RR_{B} and, when step (d) is carried out, alternatively or additionally into the at least one reaction column RD_{X}.

The introduction of the fresh alcohol ROH is effected, in particular, directly as feed stream S_{AE1} comprising ROH into the reaction column RR_{A} or, in the embodiments in which step (a2) is carried out, into the reaction columns RR_{A} and RR_{B}.

At least part of the vapour stream S_{AB} taken off at the offtake point E_{AK} at the upper end of the rectification column RD_{A} is preferably fed to the reaction column RR_{A} at least as part of the feed stream S_{AE1} and, in the cases in which step (a2) is carried out in the process of the invention, alternatively or additionally to the reaction column RR_{B} at least as part of the feed stream S_{BE1}.

In a further preferred embodiment in which step (d) is carried out, the vapour stream S_{XB} can be used at least partly as feed stream S_{AE1} in step (a1) and optionally as feed stream S_{BE1} in step (a2).

In the particularly preferred embodiment in which step (d) is carried out, S_{DAB} and S_{XB} can be used at least partly as feed stream S_{AE1} in step (a1) and optionally as feed stream S_{BE1} in step (a2). S_{DAB} and S_{XB} can then be fed separately from one another to the respective reactive rectification column RR_{A} or RR_{B} or firstly be mixed with one another and then fed to the respective reactive rectification column RR_{A} or RR_{B}. In this embodiment, S_{DAB} and S_{XB} are preferably firstly mixed with one another and then fed to the respective reactive rectification column RR_{A} or RR_{B}.

In this preferred embodiment, greater preference is given to the fresh alcohol ROH being supplied to at least one of the rectification columns RD_{A}, RD_{X}, preferably both columns RD_{A} and RD_{X}.

When the fresh alcohol ROH is supplied to the rectification column RD_{A} or RD_{X}, it is preferably fed in either in the enrichment section of the respective rectification column or directly at the top of the respective rectification column. The optimal feed point is dependent on the water content of the fresh alcohol used and secondly on the desired residual water content in the vapour stream RD_{A} or RD_{X}. The higher the proportion of water in the alcohol used and the higher the purity requirement in the vapour stream S_{DAB} or S_{XB}, the more useful is an inlet of some theoretical plates below the top of the rectification column RD_{A} or RD_{X}. Preference is given to up to 20 theoretical plates below the top of the rectification column RD_{A} or RD_{X}, and in particular from 1 to 5 theoretical plates.

When the fresh alcohol ROH is introduced into the rectification column RD_{A} or RD_{B}, it is introduced at temperatures up to the boiling point, preferably at room temperature. Introduction of the fresh alcohol ROH in gaseous form is also possible. Here, a dedicated inlet can be provided for the fresh alcohol

### 4.5 Step (d) of the Process According to the Invention (Optional)

Step (d) of the process according to the invention is optionally carried out. In the optional step (d) of the process according to the invention, S_{DAS} is, in its entirety or partly, separated in at least one rectification column RD_{X} which is different from RD_{A} into a vapour stream S_{XB} comprising ROH at the upper end of RD_{X} and a bottom stream S_{XS} comprising water and optionally ROH at the lower end of RD_{X}.

As a result, a mixture G_{X} comprising water and ROH is present in the at least one rectification column RDₓ when step (d) is carried out.

The bottom stream S_{XS} is discharged from the at least one rectification column RD_{X} at an offtake point E_{XS} at the lower end of the column and the vapour stream S_{XB} is discharged from the at least one rectification column RD_{X} at an offtake point E_{XK} at the upper end of the column.

For the purposes of the present invention, “offtake point E_{XK} at the upper end of the at least one rectification column RD_{X}” means that E_{XK} is installed on the at least one rectification column RD_{X} in such a way that the vapour stream S_{XB} is taken off from the at least one rectification column RD_{X} as overhead stream or as side offtake stream above the internals.

For the purposes of the present invention, “offtake point E_{XS} at the lower end of the at least one rectification column RD_{X}” means that E_{XS} is installed on the column in such a way that the bottom stream S_{XS} is taken off from the at least one rectification column RD_{X} as bottom stream or as side offtake stream below the internals.

The pressure pₓ in RD_{X} can be set by a person skilled in the art on the basis of such a person's specialist knowledge. It is preferably in the range from 1 bar to 20 bar, preferably from 1 bar to 15 bar, more preferably from 1 bar to 10 bar.

The temperature T_{X} in RD_{X} can be set by a person skilled in the art on the basis of such a person's specialist knowledge. It is preferably in the range from 40° C. to 220° C., preferably from 60° C. to 190° C.

The feature “at least one rectification column RD_{X} which is different from RD_{A}” means that the optional step (d) of the process of the invention encompasses the case in which the separation of the vapour stream S_{DAS} is carried out in only one rectification column RD_{X}. However, it also encompasses the case where the separation of the vapour stream S_{DAS} is carried out in more than one rectification column RD_{X}.

In the embodiment of the present invention in which more than one column RD_{X} is used for the separation of S_{DAS} and the column RD_{A} is thus followed by more than one column RD_{X}, the first of these columns RD_{X}, into which the bottom stream S_{DAS} from RD_{A} is fed, is referred to as “RD_{X1}” and the column(s) following the column RD_{X1} and into which the bottom stream from RD_{X1} is fed is/are referred to as “RD_{X2}”, etc.

Each rectification column RD_{X} which is used in addition to the first rectification column “RD_{X1}” is referred to generally as “RD_{X(n+1)}”, where n is an integer and indicates the number of the total number of rectification columns RD_{X} beyond the rectification column RD_{X1}. At the same time, the integer “n” assigned to a particular column RD_{X(n+1)} denotes the number of columns RD_{X} which are located between the column RD_{X(n+1)} concerned and RD_{A}.

Accordingly, in the case of a total of two columns RD_{X} n would be 1, in the case of three columns RD_{X} n would be 2, etc.

In the embodiments of the present invention in which more than one column RD_{X} is used for the separation of S_{DAS}, a vapour stream S_{XB1} comprising ROH is then obtained at the upper end of each of the rectification columns RD_{X1} and a bottom stream S_{XS1} comprising water and optionally ROH is obtained at the lower end of each of the rectification columns RD_{X1}. The mixture G_{X1} comprising water and ROH is present in the rectification column RD_{X1}.

In each additional rectification column RD_{X(n+1)}, a vapour stream S_{XB(n+1)} comprising ROH is then correspondingly obtained at the upper end of RD_{X(n+1)} and a bottom stream S_{XB(n+1)} comprising water and optionally ROH is obtained at the lower end of RD_{X(n+1)}. The mixture G_{X(n+1)} comprising water and ROH is present in the rectification column RDₓ₍ₙ₊₁₎. “n” here is an integer having the above-described meaning.

In the embodiment of the present invention in which more than one column RD_{X} is used for the separation of S_{DAS}, this separation is then carried out so that S_{DAS} is separated in RD_{X1} into a vapour stream S_{XB1} comprising ROH at the upper end of RD_{X1} and a bottom stream S_{XS1} comprising water and optionally ROH at the lower end of RD_{X1}. In each column RD_{X(n+1)} following RD_{X1}, the bottom stream S_{Xsn} from the preceding column RD_{Xn} is then separated into a vapour stream S_{XB(n+1)} comprising ROH at the upper end of RD_{X(n+1)} and a bottom stream S_{XS(n+1)} comprising water and optionally ROH at the lower end of RD_{X(n+1)}.

In this embodiment, too, S_{DAS} is overall separated into a vapour stream S_{XB} comprising ROH [which is then composed of the individual vapour streams S_{XB1} and S_{XB(n+1)}] and a bottom stream S_{XS} comprising water and optionally ROH [which then corresponds to the bottom stream S_{XS(n+1)} of the last column RD_{X(n+1)}].

The bottom stream S_{DAS} can, in an embodiment of the present invention, be conveyed via a pump into the at least one rectification column RD_{X}. Likewise, in the case of a plurality of rectification columns RD_{X}, a pump can be used in each case in order to convey the bottom stream S_{XSn} from a rectification column RD_{Xn} into the rectification column RD_{X(n+1)}.

Rectification columns suitable as rectification column RD_{X} are known to those skilled in the art. Preferred embodiments such as internals, etc., in RD_{X} are those described in section 4.3 for RD_{A}.

The separation in step (d) of the process of the invention is a fractional distillation as is known to a person skilled in the art of the alcohol/water mixture G_{X} which is obtained in the at least one rectification column RD_{X} when the bottom stream S_{DAS} is fed into the at least one rectification column RD_{X}.

If only one rectification column RD_{X} is used, the bottom stream S_{XS} taken off from the rectification columns RD_{X} in step (d) comprises ROH in a proportion by mass of, in particular, from 0.0001 to 3% by weight, preferably from 0.005 to 1% by weight, in each case based on the total mass of S_{XS}. S_{XS} preferably comprises essentially water in addition to the alcohol ROH.

If more than one rectification column RD_{X} is used, the bottom stream S_{XS(n+1)} which has been taken off at the bottom of the last rectification column RD_{X(n+1)} comprises ROH in a proportion by mass of, in particular, from 0.0001 to 3% by weight, preferably from 0.005 to 1% by weight, in each case based on the total mass of S_{XS}. S_{XS} preferably comprises essentially water in addition to the alcohol ROH.

At the top (another word for “top” is “at the upper end”) of the at least one rectification column RD_{X}, the vapour stream S_{XB} comprising ROH is additionally obtained. The preferred proportion by mass of ROH in this vapour stream S_{XB} is 99% by weight, preferably 99.6% by weight, more preferably 99.9% by weight, in each case based on the total mass of S_{XB}, with the balance being, in particular, water.

In an optional embodiment of the process of the invention, at least one further stream S_{XZ} which comprises ROH and water and is different from S_{XS} and S_{XB} is discharged from the at least one rectification column RD_{X} at an offtake point E_{XZ}, energy is transferred to this stream and the stream is recirculated to the same at least one rectification column RD_{X}, where the position of the offtake point E_{XZ} on the at least one rectification column RD_{A} is located between the two offtake points E_{XS} and E_{XK}. The stream S_{XZ} comprises the mixture G_{X}.

In this optional embodiment of the optional step (d) of the process of the invention, it is possible to supply energy to the mixture G_{X}. In a preferred embodiment, this occurs by the mixture G_{X} being conveyed as stream S_{XZ} via an intermediate vaporizer VZ_{X} in which energy is transferred from a heat transfer medium, in particular heating vapour H₁ or H₄ or a different heat transfer medium W₁ to S_{XZ} or G_{X}. This energy transfer can advantageously be carried out by the mixture G_{X} being conveyed as stream S_{XZ} and W₁ or the mixture G_{X} being conveyed as stream S_{XZ} and the heating vapour H₁ or H₄ being conveyed via an intermediate vaporizer VZ_{X}.

At least part of the vapour stream S_{XB} taken off at the offtake point E_{XK} at the upper end of the at least one rectification column RD_{X} is preferably fed to the reaction column RR_{A} at least as part of the feed stream S_{AE1} and, in the cases in which step (a2) is carried out in the process of the invention, alternatively or additionally to the reaction column RR_{B} at least as part of the feed stream S_{BE1}.

When both S_{DAB} and also S_{XB} are introduced at least as part of the feed stream S_{AE1} and, in the cases in which step (a2) is carried out in the process of the invention, alternatively or additionally fed to the reaction column RR_{B} at least as part of the feed stream S_{BE1}, these can be mixed beforehand and then fed as a feed stream S_{AE1} or S_{BE1} into the respective reaction column RR_{A} or RR_{B}, or they are fed as separate streams into the respective reaction column RR_{A} or RR_{B}.

### 4.6 Steps (e) and (h) of the Process According to the Invention

In step (e), energy, preferably heat, is transferred from a heating vapour H₁ having a pressure p₁ to at least one component Q₁, as a result of which the heating vapour H₁ condenses at least partially and a condensate K₁ is thus obtained.

In step (h), energy is transferred from a heating vapour H₄ having a pressure p₄ to at least one component Q₂, as a result of which, in particular, the heating vapour H₄ condenses at least partially and a condensate K₂ is thus obtained.

For the purposes of the invention, any vapour stream can be used as heating vapour H₁ or H₄. For the purposes of the invention, it can, for example, comprise n-butane or water. It is preferably steam.

“Component Q₁” and “component Q₂” are, according to the invention, collective terms for the mixtures and crude products S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS}.

In other words, Q₁ is selected from the group consisting of S_{AE1}, S_{AE2}, RP_{A}, S_{AS}, S_{AB}, G_{A}, S_{DAS}, S_{DAB}, and can, when step (a2) is carried out, alternatively or additionally be selected from the group consisting of S_{BE1}, S_{BE2}, RP_{B}, S_{BS}, S_{BB}, and can also, when step (d) is carried out, alternatively or additionally be selected from the group consisting of G_{X}, S_{XB}, S_{XS}.

Q₂ can be selected correspondingly: Q₂ is selected from the group consisting of S_{AE1}, S_{AE2}, RP_{A}, S_{AS}, S_{AB}, G_{A}, S_{DAS}, S_{DAB}, and can, when step (a2) is carried out, alternatively or additionally be selected from the group consisting of S_{BE1}, S_{BE2}, RP_{B}, S_{BS}, S_{BB}, and can also, when step (d) is carried out, alternatively or additionally be selected from the group consisting of G_{X}, S_{XB}, S_{XS}.

This means that in the embodiments of the process of the invention in which the step (a2) is carried out but step (d) is not carried out, the component Q₁ is selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB},

and in the embodiments of the process of the invention in which step (d) is carried out but step (a2) is not carried out, the component Q₁ is selected from S_{AE1}, S_{AE2}, RP_{A}, S_{AS}, S_{AB}, G_{A}, S_{DAS}, S_{DAB}, G_{X}, S_{XB}, S_{XS},
and in the embodiments of the process of the invention in which both steps (a2) and (d) are carried out, the component Q₁ is selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS}.

This means that in the embodiments of the process of the invention in which step (a2) is carried out but step (d) is not carried out, the component Q₂ is selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB},

and in the embodiments of the process of the invention in which step (d) is carried out but step (a2) is not carried out, the component Q₂ is selected from S_{AE1}, S_{AE2}, RP_{A}, S_{AS}, S_{AB}, G_{A}, S_{DAS}, S_{DAB}, G_{X}, S_{XB}, S_{XS},
and in the embodiments of the process of the invention in which both steps (a2) and (d) are carried out, the component Q₂ is selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS}.

Q₁ and Q₂ are identical or different and are preferably identical.

When Q₁ and Q₂ are identical, this means, according to the invention, that the mixture or the stream selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS} to which energy is transferred from heating vapour H₁ in step (e) is identical to the mixture or the stream selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS} to which energy is transferred from heating vapour H₄ in step (h).

When Q₁ and Q₂ are different, this means, according to the invention, that the mixture or the stream selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS} to which energy is transferred from heating vapour H₁ in step (e) is different from the mixture or the stream selected from S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS} to which energy is transferred from heating vapour H₄ in step (h).

In the embodiments of the present invention in which more than one column RD_{X} is used for separating S_{DAS}, “S_{XS}” designates the group consisting of the bottom stream S_{XS1} and the n bottom streams S_{XS(n+1)}, where “S_{XS(n+1)}” is the respective bottom stream from the rectification column RD_{X(n+1)}.

In the embodiments of the present invention in which more than one column RD_{X} is used for separating S_{DAS}, “S_{XB}” designates the group consisting of the vapour stream S_{XB1} and the n vapour streams S_{XB(n+1)}, where “S_{XB(n+1)}” is the respective vapour stream from the rectification column RD_{X(n+1)}.

In the embodiments of the present invention in which more than one column RD_{X} is used for separating S_{DAS}, “G_{X}” designates the group consisting of the mixture G_{X1} and the n mixtures G_{X(n+1)}, where “G_{X(n+1)}” is the respective mixture G_{X} in the rectification column RD_{X(n+1)}.

The at least one component Q₁ is, in a preferred embodiment of the present invention, selected from the group consisting of S_{AE1}, S_{AE2}, RP_{A}, S_{AS}, G_{A}, S_{DAS},
where, when step (a2) is carried out, the at least one component Q₁ can alternatively or additionally be selected from S_{BE1}, S_{BE2}, RP_{B}, S_{BS},
and where, when step (d) is carried out, the at least one component Q₁ can alternatively or additionally be selected from G_{X}, S_{XS}.

In a more preferred embodiment of the present invention, the at least one component Q₁ is selected from the group consisting of RP_{A}, S_{AS}, G_{A}, S_{DAS},
where, when step (a2) is carried out, the at least one component Q₁ can alternatively or additionally be selected from RP_{B}, S_{BS},
and where, when step (d) is carried out, the at least one component Q₁ can alternatively or additionally be selected from G_{X}, S_{XS}.

In an even more preferred embodiment of the present invention, the at least one component Q₁ is selected from the group consisting of G_{A}, S_{DAS},
and where, when step (d) is carried out, the at least one component Q₁ can alternatively or additionally be selected from G_{X}, S_{XS}.

In an even more preferred embodiment of the present invention, the at least one component Q₁ is selected from the group consisting of G_{A}, S_{DAS}.

In a preferred embodiment of the present invention, the at least one component Q₂ is selected from the group consisting of S_{AE1}, S_{AE2}, RP_{A}, S_{AS}, G_{A}, S_{DAS},
where, when step (a2) is carried out, the at least one component Q₂ can alternatively or additionally be selected from S_{BE1}, S_{BE2}, RP_{B}, S_{BS},
and where, when step (d) is carried out, the at least one component Q₂ can alternatively or additionally be selected from G_{X}, S_{XS}.

In a more preferred embodiment of the present invention, the at least one component Q₂ is selected from the group consisting of RP_{A}, S_{AS}, G_{A}, S_{DAS},
where, when step (a2) is carried out, the at least one component Q₂ can alternatively or additionally be selected from RP_{B}, S_{BS},
and where, when step (d) is carried out, the at least one component Q₂ can alternatively or additionally be selected from G_{X}, S_{XS}.

In a further preferred embodiment of the present invention, the at least one component Q₂ is selected from the group consisting of G_{A}, S_{DAS},

and where, when step (d) is carried out, the at least one component Q₂ can alternatively or additionally be selected from G_{X}, S_{XS}.

In an even more preferred embodiment of the present invention, the at least one component Q₂ is selected from the group consisting of G_{A}, S_{DAS}.

The energy of the heating vapour H₁ or H₄ can be transferred to the at least one component Q₁ or Q₂ in step (e) or step (h) of the process of the invention by methods known to those skilled in the art. In particular, a heat exchanger WT is used for this purpose. All heat exchangers which are well known to a person skilled in the art for this purpose can be utilized as heat exchanger WT (also referred to as “heat exchanger”). This is preferably a vaporizer, more preferably selected from bottom vaporizer VS, intermediate vaporizer VZ.

The transfer of energy from the heating vapour H₁ or H₄ to the at least one component Q₁ or Q₂ is, according to the invention, effected in particular by heat being transferred from the heating vapour H₁ or H₄ to the at least one component Q₁ or Q₂, so that the heating vapour H₁ or H₄ heats the at least one component Q₁ or Q₂.

The transfer of energy from the heating vapour H₁ to the at least one component Q₁ occurs in particular, directly or indirectly.

The transfer of energy from the heating vapour H₄ to the at least one component Q₂ occurs, in particular, directly or indirectly.

In the case of H₁ and Q₁, “directly” means that H₁ is contacted with the at least one component Q₁ so that energy, preferably heat, is transferred from H₁ to the at least one component Q₁. Contacting here preferably takes place in a heat exchanger WT, even more preferably in a vaporizer.

In the case of H₄ and Q₂, “directly” means that H₄ is contacted with the at least one component Q₂, so that energy, preferably heat, is transferred from H₄ to the at least one component Q₂. The contacting here preferably takes place in a heat exchanger WT, even more preferably in a vaporizer.

It goes without saying that both in the case of “direct” and in the case of “indirect” energy transfer, H₁ and Q₁ or H₄ and Q₂ or H₁ or H₄ and W₁ do not mix since no condensate K₁ or K₂ would then be obtained. The contacting without mixing is achieved by methods known to those skilled in the art, for example by contacting via a dividing wall composed of metal, polymer, etc., in particular in the heat exchanger WT.

In the case where Q₁ or Q₂ is S_{AE1}, the direct energy transfer from H₁ or H₄ to S_{AE1} is carried out, in particular, by S_{AE1} and H₁ or H₄ being conveyed through a heat exchanger WT (e.g. <WA4> in FIGS. 1 to 4) before S_{AE1} is introduced into the reaction column RR_{A}, so that S_{AE1} contacts the heating vapour H₁ or the heating vapour H₄ in the heat exchanger WT and energy is thus transferred from H₁ or H₄ to S_{AE1}.

In the case where Q₁ or Q₂ is S_{AE2}, the direct energy transfer from H₁ or H₄ to S_{AE2} is, in particular, carried out by S_{AE2} and H₁ or H₄ being conveyed through a heat exchanger WT (e.g. <WA5> in FIGS. 1 to 4) before S_{AE2} is introduced into the reaction column RR_{A}, so that S_{AE2} contacts the heating vapour H₁ or the heating vapour H₄ in the heat exchanger WT and energy is thus transferred from H₁ or H₄ to S_{AE2}.

In the case where Q₁ or Q₂ is RP_{A}, the direct energy transfer from H₁ or H₄ to RP_{A} is, in particular, carried out by RP_{A} being discharged as stream S_{RRAZ} from the column RR_{A} and S_{RRAZ} and H₁ or H₄ being conveyed through a heat exchanger WT, preferably an intermediate vaporizer VZ_{RRA} (e.g. <WA3> in FIGS. 1 to 4) on the reaction column RR_{A}, so that S_{RRAZ} contacts the heating vapour H₁ or the heating vapour H₄ in the heat exchanger WT and energy is thus transferred from H₁ or H₄ to S_{RRAZ}. S_{RRAZ} is then recirculated to the other RP_{A} in RR_{A}.

In the case where Q₁ or Q₂ is S_{AS}, the direct energy transfer from H₁ or H₄ to S_{AS} is, in particular, carried out by S_{AS} and H₁ or H₄ being conveyed through a heat exchanger WT, preferably a bottom vaporizer VS_{RRA} (e.g. <WA1> in FIGS. 1 to 4) on the reaction column RR_{A}, so that S_{AS} contacts the heating vapour H₁ or the heating vapour H₄ in the heat exchanger WT and energy is thus transferred from H₁ or H₄ to S_{AS}.

In the case where Q₁ or Q₂ is S_{AB}, the direct energy transfer from H₁ or H₄ to S_{AB} is, in particular, carried out by S_{AB} and H₁ or H₄ being conveyed through a heat exchanger WT (e.g. <WA6> in FIGS. 1 to 4), so that S_{AB} contacts the heating vapour H₁ or the heating vapour H₄ in the heat exchanger WT and energy is thus transferred from H₁ or H₄ to S_{AB}.

In the case where Q₁ or Q₂ is G_{A}, the direct energy transfer from H₁ or H₄ to G_{A} is, in particular, carried out by G_{A} being discharged as stream S_{AZ} from the column RR_{A} and S_{AZ} and H₁ or H₄ being conveyed through a heat exchanger WT, preferably an intermediate vaporizer VZ_{A} (e.g. <W9> in FIGS. 1 to 4) on the rectification column RD_{A}, so that S_{AZ} contacts the heating vapour H₁ or the heating vapour H₄ in the heat exchanger WT and energy is thus transferred from H₁ or H₄ to S_{AZ}. S_{AZ} is then recirculated to the other G_{A} in RD_{A}.

In the case where Q₁ or Q₂ is S_{DAS}, the direct energy transfer from H₁ or H₄ to S_{D}AS is, in particular, carried out by S_{DAS} and H₁ or H₄ being conveyed through a heat exchanger WT, preferably a bottom vaporizer VS_{A} (e.g. <W7> in FIGS. 1 to 4) to the rectification column RD_{A}, so that S_{D}AS contacts the heating vapour H₁ or the heating vapour H₄ in the heat exchanger WT and energy is thus transferred from H₁ or H₄ to S_{DAS}.

In the case where Q₁ or Q₂ is S_{DAB}, the direct energy transfer from H₁ or H₄ to S_{DAB} is, in particular, carried out by S_{DA}B and H₁ or H₄ being conveyed through a heat exchanger WT (not shown in the figures), so that S_{DAB} contacts the heating vapour H₁ or the heating vapour H₄ in the heat exchanger WT and energy is thus transferred from H₁ or H₄ to S_{DAB}.

In the case where Q₁ or Q₂ is S_{BE1}, the direct energy transfer from H₁ or H₄ to S_{BE1} is, in particular, carried out by S_{BE1} and H₁ or H₄ being conveyed through a heat exchanger WT (e.g. <WB4> in FIG. 4) before S_{BE1} is introduced into the reaction column RR_{B}, so that S_{BE1} contacts the heating vapour H₁ or the heating vapour H₄ in the heat exchanger WT and energy is thus transferred from H₁ or H₄ to S_{BE1}.

In the case where Q₁ or Q₂ is S_{BE2}, the direct energy transfer from H₁ or H₄ to S_{BE2} is, in particular, carried out by S_{BE2} and H₁ or H₄ being conveyed through a heat exchanger WT (e.g. <WBA5> in FIG. 4) before S_{BE2} is introduced into the reaction column RR_{B}, so that S_{BE2} contacts the heating vapour H₁ or the heating vapour H₄ in the heat exchanger WT and energy is thus transferred from H₁ or H₄ to S_{BE2}.

In the case where Q₁ or Q₂ is RP_{B}, the direct energy transfer from H₁ or H₄ to RP_{B} is, in particular, carried out by RP_{B} being discharged as stream S_{RRBZ} from the column RR_{B} and S_{RRBZ} and H₁ or H₄ being conveyed through a heat exchanger WT, preferably an intermediate vaporizer VZ_{RRB} (e.g. <WB3> in FIG. 4) on the reaction column RR_{B}, so that S_{RRBZ} contacts the heating vapour H₁ or the heating vapour H₄ in the heat exchanger WT and energy is thus transferred from H₁ or H₄ to S_{RRBZ}. S_{RRBZ} is then recirculated to the other RP_{B} in RR_{B}.

In the case where Q₁ or Q₂ is S_{BS}, the direct energy transfer from H₁ or H₄ to S_{BS} is, in particular, carried out by S_{B}S and H₁ or H₄ being conveyed through a heat exchanger WT, preferably a bottom vaporizer VS_{RRB} (e.g. <WB1> in FIG. 4) on the reaction column RR_{B}, so that S_{B}S contacts the heating vapour H₁ or the heating vapour H₄ in the heat exchanger WT and energy is thus transferred from H₁ or H₄ to S_{BS}.

In the case where Q₁ or Q₂ is S_{BB}, the direct energy transfer from H₁ or H₄ to S_{BB} is, in particular, carried out by S_{BB} and H₁ or H₄ being conveyed through a heat exchanger WT (e.g. <WB6> in FIG. 4), so that S_{BB} contacts the heating vapour H₁ or the heating vapour H₄ in the heat exchanger WT and energy is thus transferred from H₁ or H₄ to S_{BB}.

In the case where Q₁ or Q₂ is G_{X}, the direct energy transfer from H₁ or H₄ to G_{X} is, in particular, carried out by G_{X} being discharged as stream S_{XZ} from the column RD_{X} and S_{XZ} and H₁ or H₄ being conveyed through a heat exchanger WT, preferably an intermediate vaporizer VZ_{X} (e.g. <W13> in FIG. 4) on the rectification column RD_{X}, so that S_{XZ} contacts the heating vapour H₁ or the heating vapour H₄ in the heat exchanger WT and energy is thus transferred from H₁ or H₄ to S_{XZ}. S_{XZ} is then recirculated to the other G_{X} in RD_{X}.

In the case where Q₁ or Q₂ is S_{XS}, the direct energy transfer from H₁ or H₄ to S_{XS} is, in particular, characterized by S_{XS} and H₁ or H₄ being conveyed through a heat exchanger WT, preferably a bottom vaporizer VS_{X} (e.g. <W11> in FIG. 4) on the rectification column RD_{X}, so that S_{XS} contacts the heating vapour H₁ or the heating vapour H₄ in the heat exchanger WT and energy is thus transferred from H₁ or H₄ to S_{XS}.

In the case where Q₁ or Q₂ is S_{XB}, the direct energy transfer from H₁ or H₄ to S_{XB} is, in particular, characterized by S_{XB} and H₁ or H₄ being conveyed through a heat exchanger WT (not shown in the figures), so that S_{XB} contacts the heating vapour H₁ or the heating vapour H₄ in the heat exchanger WT and energy is thus transferred from H₁ or H₄ to S_{XB}.

In the case of H₁ and Q₁, “indirect” means that energy is transferred from heating vapour H₁ to a heat transfer medium W₁, in particular H₁ is contacted with a heat transfer medium W₁, so that energy, preferably heat, is transferred from H₁ to W₁ and the energy of W₁ is subsequently transferred to the at least one component Q₁. The transfer of the energy from W₁ to Q₁ can in turn take place directly or indirectly, i.e. by W₁ being contacted with the at least one component Q₁, so that energy, preferably heat, of W₁ goes over (“directly”) to the at least one component Q₁ or so that the energy of W₁ is firstly transferred to one or more further heat transfer media W₂, W₃, W₄ etc., and the last of these heat exchangers, designated as “W_{Y}” is contacted with the at least one component Q₁, so that energy, preferably heat, goes over from W_{Y} to the at least one component Q₁. The contacting described preferably takes place in each case in a heat exchanger WT, even more preferably in a vaporizer.

In the case of indirect energy transfer from H₁ to the at least one component Q₁, preference is given to H₁ being contacted with a heat transfer medium W₁ so that energy, preferably heat, goes over from H₁ to W₁ and the energy of W₁ is subsequently transferred to Q₁ by contacting of W₁ with the at least one component Q₁ in a heat exchanger WT.

The transfer of energy from W₁ to Q₁ can, in the embodiment in which W₁ is a stream comprising essentially water and/or the alcohol ROH, also be carried out by W₁ being mixed with the at least one component Q₁ after energy has been transferred from heating vapour H₁ to W₁, preferably in a heat exchanger WT. This embodiment is particularly advantageous when the at least one component Q₁ is selected from the group consisting of RP_{A}, G_{A}, RP_{B}, G_{X}, preferably from G_{A}, G_{X}.

“Stream comprising essentially water and/or alcohol ROH” means, in particular, that the proportion by mass of alcohol ROH and water in the stream is 90% by weight, preferably 96% by weight or 99% by weight. Even more preferably, it means that the ratio of the mass of water comprised by the stream to alcohol ROH comprised by the stream is less than 1:1, preferably 1:5, more preferably 1:9, more preferably 1:49, even more preferably 1:99, even more preferably 1:999.

In the case of H₄ and Q₂, “indirect” means that energy is transferred from heating vapour H₄ to a heat transfer medium W₁, in particular H₄ is contacted with a heat transfer medium W₁, so that energy, preferably heat, is transferred from H₄ to W₁ and the energy of W₁ is subsequently transferred to the at least one component Q₂. The transfer of energy from W₁ to Q₂ can in turn take place directly or indirectly, i.e. by W₁ being contacted with the at least one component Q₂ so that energy, preferably heat, goes over (“directly”) from W₁ to the at least one component Q₂ or so that the energy of W₁ is firstly transferred to one or more further heat transfer media W₂, W₃, W₄ etc., and the last of these heat transfer media, designated as “W_{Y}” is contacted with the at least one component Q₂ so that energy, preferably heat, goes over from W_{Y} to the at least one component Q₂. The contacting events described in each case preferably take place in a heat exchanger, even more preferably in a vaporizer.

In the case of indirect energy transfer from H₄ to the at least one component Q₂, preference is given to H₄ being contacted with a heat transfer medium W₁, so that energy, preferably heat, goes over from H₄ to W₁ and the energy of W₁ is subsequently transferred to Q₂ by contacting of W₁ with the at least one component Q₂ in a heat exchanger WT.

In the embodiment in which W₁ is a stream comprising essentially water and/or the alcohol ROH, the indirect transfer of energy from W₁ to the at least one component Q₂ is preferably carried out so that W₁ is mixed with the at least one component Q₂ after energy has been transferred from the heating vapour H₁ to W₁, preferably in a heat exchanger WT. This embodiment is particularly advantageous when the at least one component Q₂ is selected from the group consisting of RP_{A}, G_{A}, RP_{B}, G_{X}, preferably from G_{A}, G_{X}.

The mixing of W₁ with the at least one component Q₁ or the at least one component Q₂ is, in particular, carried out by W₁, for example when the component Q₁ or Q₂ is a stream S_{AE1}, S_{AE2}, S_{AS}, S_{AB}, S_{DAS}, S_{DAB}, S_{BE1}, S_{BE2}, S_{BS}, S_{BB}, S_{XB}, S_{XS}, being introduced into the respective stream in the conduit. When the component Q₁ or Q₂ is one of the mixtures RP_{A}, G_{A}, RP_{B}, G_{X}, W₁ can advantageously be introduced into the column in which the mixture concerned is present, thus in the case of RP_{A} in RR_{A}, in the case of RP_{B} in RR_{B}, in the case of G_{A} in RD_{A} and in the case of G_{X} in RD_{X}.

According to the invention, W₁ and all heat transfer media W₂, W₃, W₄ etc., which may be used in indirect energy transfer are none of S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS}.

This also encompasses, according to the invention, the case where W₁, W₂, W₃, W₄ etc., comprises the same material as one of S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS} and differs only from the stream or the mixture in terms of the proportion by mass of the material.

Any heat transfer medium known to those skilled in the art can be utilized as heat transfer medium W₁ and optionally further heat transfer media W₂, W₃, W₄ etc., employed. The heat transfer media are preferably selected from the group consisting of water; alcohols, preferably the alcohol ROH; alcohol-water solutions, preferably ROH-water solutions; salt-water solutions, including ionic liquids such as LiBr solutions, dialkylimidazolium salts such as, in particular, dialkylimidazolium dialkylphosphates; mineral oils such as diesel oils; heat transfer oils such as silicone oils, biological oils such as limonene; aromatic hydrocarbons such as dibenzyl toluene. The greatest preference is given to using water, alcohol or alcohol-water solutions as heat transfer medium, in particular as heat transfer medium W₁, where the alcohol is preferably the alcohol ROH and the alcohol is the same alcohol as is also used in step (a1) and optionally in step (a2).

Salt-water solutions that may be used are also described for example in DE 10 2005 028 451 A1 and WO 2006/134015 A1.

This embodiment in which energy is transferred indirectly is accordingly particularly advantageous when fresh alcohol ROH is to be introduced into the system.

In the context of the invention, “bar” always means “bar absolute” (=“bar abs.”).

H₁ has, in particular, a temperature T₁ of from 101° C. to 270° C., preferably from 105° C. to 250° C., preferably from 130° C. to 200° C. H₁ preferably has a pressure of from 1 to 55 bar, more preferably from 2 to 5 bar, even more preferably 4 bar. As heating vapour H₁, it is also possible to use a vapour having a pressure of >30 bar.

The heating vapour H₁ condenses at least partially on transfer of the energy, as a result of which a condensate K₁ is obtained at a pressure of from 1 to 55 bar, preferably from 2 to 5 bar, particularly preferably 4 bar, and a temperature of from 101° C. to 270° C., preferably from 105° C. to 250° C., more preferably from 110° C. to 200° C.

The heating vapour condensate K₁ is preferably collected in a condensate vessel. All vessels known to those skilled in the art, e.g. steel vessels, can be utilized for the purpose.

### 4.7 Step (f) of the Process According to the Invention

In step (f) of the process according to the invention, K₁ is at least partially depressurized to give a heating vapour H₂ having a pressure p₂ and a temperature T₂, where p₂<p₁.

The depressurization of the heating vapour condensate K₁ is preferably carried out by introducing K₁ into a condensate vessel in which the hot condensate can be separated from the vapour. In a preferred embodiment of the process of the invention, K₁ is utilized for heating streams and/or mixtures in the process before it is depressurized to give a heating vapour H₂. Likewise, the remaining condensate which remains in the liquid state after depressurization to p₂ can be utilized for heating streams and/or mixtures in the process.

The pressure in the condensate vessel is preferably lower than in the heat exchanger (in which H₁ is condensed at least partially) on the heating vapour side. Owing to the lower pressure, part of the hot condensate K₁ can vaporize, as a result of which the entire vapour, i.e. the uncondensed part of the heating vapour and the hot condensate which has been vaporized by depressurization in the condensate vessel is obtained as heating vapour H₂ in the condensate vessel. This heating vapour H₂ is, in particular, low-pressure vapour and preferably has a pressure p₂ which is such that the ratio p₂/p₁ (bar/bar) is in the range from 0.001 to 0.999, preferably from 0.01 to 0.9, more preferably from 0.05 to 0.8, more preferably from 0.1 to 0.7, more preferably from 0.15 to 0.6, more preferably from 0.2 to 0.5.

In another preferred embodiment, the temperature T₂ of H₂ is below the temperature T₁ of H₁. It is then preferred that T₂ is >0° C., and the ratio of temperature T₂/T₁ (° C./° C.) is in the range from 0.1 to 0.999, more preferably from 0.2 to 0.995, more preferably from 0.3 to 0.990.

The condensate K₁ can be composed of not only the condensate obtained from the condensation of H₁ but also further heating vapour condensates, for instance when various heating vapours are used in the process of the invention and energy is thus transferred to various components Q₁ from different heating vapours. In this embodiment, a plurality of condensates K₁ are then obtained and these can be combined and depressurized in step (f) to give heating vapour H₂.

Such a preferred embodiment is shown, for example, in FIG. 3. The condensate K₁ collected in the condensate vessel <86> is a mixture of the condensates <71> and <81> and additionally <88>. The condensate <81> is obtained from the condensation of the heating vapour <84> on transfer of energy to G_{A} in the intermediate vaporizer VZ_{A}<W9>. The condensate <71> is obtained from the condensation of the heating vapour <74> on transfer of energy to G_{A} in the bottom vaporizer VS_{A}<W8>.

The hot condensates from these two vaporizers are then conveyed according to the above statements to a condensate vessel <86>. The heating vapour H₂<82> obtained there is then used in the regulatable vapour ejector <89>, the mixed vapour from which is reused as new heating vapour H₄<84> in the intermediate vaporizer VZ_{A}<W9>. The advantage of this variant is that the hot condensate obtained can be depressurized further in order to be able to provide a greater amount of low-pressure vapour.

FIG. 4 shows a corresponding routing of the condensate and heating vapour streams on the rectification column RD_{X}<3>.

### 4.8 Step (a) of the Process According to the Invention

In step (g) of the process of the invention, H₂ is mixed with further heating vapour H₃ having a pressure p₃ and a temperature T₃ so as to give a mixed heating vapour H₄ having a pressure p₄ and a temperature T₄, where p₂<p₄<p₃ and in particular p₁<p₃.

The condensate K₁ and thus the heating vapour H₂ still contains energy which is not exploited in any known process for producing alkali metal alkoxides. However, this is not sensible from an energy and economic point of view. This energy can be utilized in the process of the present invention.

The mixing of H₂ with further heating vapour H₃ can be carried out by any method known to those skilled in the art, in particular by combining the two heating vapours by means of a Venturi nozzle.

In a preferred embodiment of the process of the invention, the vapour H₂ is mixed with further vapour having a pressure p<p₃ and the vapour obtained then is mixed with H₃.

The mixing of H₂ with further heating vapour H₃ is preferably carried out by means of a, preferably regulatable, vapour ejector DS (thermocompressor). The thermocompressor is then supplied both with further heating vapour H₃, which originates from an appropriate vapour network, and also with the low-pressure vapour H₂ from the condensate vessel, forming a mixed vapour H₄ which is accordingly the heat transfer medium. The mixed vapour in this embodiment is accordingly the heating vapour H₄.

Such a vapour ejector DS is shown in FIG. 5. It is configured so that it is operated using a driving vapour having a relatively high pressure and can draw in a suction vapour having a lower pressure, as a result of which a mixed vapour which is used as heat transfer medium is formed. The driving vapour is in the present case the heating vapour or the driving vapour H₃ by means of which the heating vapour H₂ is drawn as suction vapour from the condensate vessel and mixed with the driving vapour H₃.

The advantage of such a configuration is obvious. Part of the energy of the low-pressure vapour obtained in the condensate vessel can be utilized and energy and costs can thus be saved. Such a procedure can also be advantageous for another reason. The vapour ejector used can be regulatable so that the amounts of intermediate-pressure vapour H₃ and low-pressure vapour H₂ can be adjusted, for example as a function of particular process parameters. The amount of suction vapour is established via the amount of driving vapour. The amount of intermediate-pressure vapour H₃ and low-pressure vapour H₂ can, for example, be set as a function of the temperature of the at least one component Q₁.

The pressure p₃ of the heating vapour H₃ is greater than p₂ and can otherwise be selected in a wide range. The invention is suitable for situations in which heating vapour H₃ on whose pressure and temperature little influence can be exerted and which then has to be set to a given value H₄ arises. Thus, p₂<p₄<p₃.

In a particular embodiment of the process of the invention, p₂<p₁<p₃, especially when Q₁ and Q₂ are identical.

The heating vapour H₃ used preferably has a pressure p₃ in the range from 2 bar to 80 bar and preferably has a temperature T₃ of from 120° C. to 300° C., where at the same time p₄<p₃ and preferably also p₁<p₃.

### 4.9 Step (h) of the Process According to the Invention

In step (h), energy is transferred from heating vapour H₄ having a pressure p₄ and a temperature T₄ to at least one component Q₂.

The meaning of the component Q₂ is defined above in the context of step (e), section 4.6.

In a preferred embodiment of the present invention, the heating vapour H₄ condenses at least partially and a condensate K₂ is thus obtained.

H₄ has, in particular, a temperature T₄ of from 101° C. to 270° C., preferably from 105° C. to 250° C., more preferably from 110° C. to 200° C. H₄ preferably has a pressure of from 1 to 55 bar, preferably from 2 to 5 bar, more preferably 4 bar. As heating vapour H₄, it is also possible to use a vapour having a pressure of >30 bar.

The heating vapour H₄ condenses at least partially, in particular, on transfer of the energy, as a result of which a condensate K₂ is preferably obtained at a pressure of from 1 to 55 bar, preferably from 2 to 5 bar, particularly preferably 4 bar, and a temperature of from 101° C. to 270° C., preferably from 105° C. to 250° C., more preferably from 110° C. to 200° C.

The heating vapour condensate K₂ is preferably collected in a condensate vessel. For this purpose, it is possible to utilize all condensate vessels known to those skilled in the art, e.g. vessels made of steel.

The advantage of the present invention is, inter alia, that the sequence of steps (f), (g), (h) can be carried out in a cycle and the condensate K₂ obtained after step (h) by the at least partial condensation of H₄ is reused as condensate K₁ in a new cycle of step (f), (g), (h).

This is advantageous especially when Q₁ and Q₂ are identical.

In a preferred embodiment of the present invention, the heating vapour H₄ is therefore at least partially condensed so as to give a condensate K₂ by means of which a further step (f) in which the condensate K₂ is then used as condensate K₁ is carried out after step (h).

This allows the process of the invention to be carried out continuously and the heating of the corresponding components Q₁ and Q₂ to be carried out in the circuit.

In the process of the invention, p₁ and p₄, are preferably similar, i.e. the ratio of p₁/p₄ is preferably in the range from 0.5 to 1.5, more preferably from 0.8 to 1.2, more preferably from 0.9 to 1.1, even more preferably from 0.99 to 1.01, most preferably 1.

### 5. EXAMPLES

### 5.1 Example (not According to the Invention)

Comparative Example 1 is carried out in an apparatus corresponding to FIG. 1.

A stream of aqueous NaOH S_{AE2}<1A2> (48.5% by weight of NaOH, 51.5% by weight of water) of 459 kg/h is fed in at room temperature at the top of a reaction column RR_{A}<1A> having a pressure at the top of 1.6 bar abs.

A methanol stream S_{AE1}<1A1> in vapour form having a low level of contamination with water is introduced in countercurrent at a rate of 6962 kg/h at the bottom of the reaction column <1A>. A methanol/water mixture S_{AB}<1A5> is obtained at the top of the reaction column and of this 551 kg/h are condensed by means of a condenser and recirculated to the column <1A> (not shown in the figures) and 6421 kg/h are conveyed further via a compressor VD₃₁<11> to the column RD_{A}<2>.

At the bottom of the column RR_{A}<1A>, a virtually water-free product stream S_{AS}<1A4> is discharged from the column. Of this, 1000 kg/h are discharged as product stream from the process. The other part is fed to the bottom vaporizer V_{SRRA}<WA1>. 294 kg/h of heating vapour (11 bar abs., 190° C.) are depressurized through a regulating valve <85> to 4 bar abs. and energy is thus transferred to this part of the stream S_{AS}<1A4> taken off via the bottom vaporizer VS_{RRA}<WA1>, resulting in the heating vapour condensing and the product stream S_{AS}<1A4> taken off partially vaporizing and the desired MeOH content thus being established in the bottom product S_{AS}<1A4>. The uncondensed part of the overhead stream S_{AB}<1A5> (6421 kg/h of MeOH/water mixture) is conveyed via the compressor VD₃₁<11> (exit pressure 2.25 bar abs.) into the rectification column RD_{A}<2> (pressure at the top of column RD_{A}<2>=2.2 bar abs.).

At the top of the rectification column RD_{A}<2>, a methanol/water mixture S_{DAB}<24> which is enriched in methanol compared to the feed stream from the compressor VD₃₁<11> is obtained, and 878 kg/h of fresh methanol <25> are additionally fed to the column RD_{A}<2> at the top of the column.

At the bottom of the rectification column RD_{A}<2>, a stream S_{DAS}<22> which is enriched in water compared to the column feed from the compressor is obtained. Part of this stream is recirculated via the bottom vaporizer VS_{A}<W8> to the column <2>, with the remaining 337 kg/h being discharged as bottom product stream.

For operation of the bottom vaporizer VS_{A}<W8>, 3256 kg/h of heating vapour (11 bar abs., 190° C.) are depressurized through a regulating valve <85> to 4 bar abs. and energy is transferred to the recirculated stream via the bottom vaporizer VS_{A}<W8>, resulting in the heating vapour condensing.

The heating vapour condensate streams from <WA1> and <W8> are combined in a condensate vessel <86>. For recirculation into the condensate network, the condensate stream <87> exiting at about 143° C. is cooled to 75° C. by means of return cooling water. Here, about 285 kW are transferred to the return cooling water.

### 5.2 Example 2 (According to the Invention)

Example 2 is carried out in an apparatus corresponding to FIG. 2.

This example is a repetition of Example 1 with the following differences: The collected condensate in the condensate vessel <86> is depressurized to 1.5 bar abs. This results in vaporization of about 217 kg/h of condensate <82>. The heating vapour <83> is not depressurized through a regulating valve <85> but instead is used as driving vapour for a regulatable vapour ejector <89> in order to draw in the stream <82> and mix it with <83>. The heating vapour <84> having a pressure of 4 bar abs. is obtained at the exit of <89>. As a result of the additional heating vapour from the depressurization of the condensate being utilized, only 3048 kg/h of fresh heating vapour <83> are acquired for operation of the bottom vaporizer VS_{A}<W8>.

A stream <84> (about 3265 kg/h) is obtained and this condenses as condensate <81> during operation of the bottom vaporizer VS_{A}<W8>. The resulting condensate stream <81> is then fed back into the condensate vessel <86>.

The condensate stream <87> is obtained at a temperature of only about 111° C. Thus, less return cooling water than in Example 1 is required for cooling to 75° C. and only about 142 kW are transferred to the cooling medium, i.e. only 50% of the amount required in Example 1.

In total, only 3342 kg/h of heating vapour (11 bar abs, 190° C.) are required as heating vapour for <WA1> and <WA8> in the procedure according to Example 2. Thus, about 6% of the 11 bar abs. heating vapour can be saved compared to Example 1.

1-15. (canceled)

16. A process for producing at least one alkali metal alkoxide of the formula M_{A}OR, wherein R is a C₁-C₆ hydrocarbon radical and M_{A} is sodium or potassium, comprising:
(a1) reacting a feed stream S_{AE1} comprising ROH with a feed stream S_{AE2} comprising M_{A}OH in countercurrent in a reactive rectification column RR_{A} to give a crude product mixture RP_{A} comprising M_{A}OR, water, ROH, and M_{A}OH,
wherein a bottom product stream S_{AS} comprising ROH and M_{A}OR is taken off at the lower end of RR_{A} and a vapour stream S_{AB} comprising water and ROH is taken off at the upper end of RR_{A},
(a2) optionally, simultaneously with, and spatially separately from step (a1), a feed stream S_{BE1} comprising ROH is reacted with a feed stream S_{BE2} comprising M_{B}OH in countercurrent in a reactive rectification column RR_{B} to give a crude product mixture RP_{B} comprising M_{B}OR, water, ROH, M_{B}OH, where M_{B} is selected from sodium or potassium,
wherein a bottom product stream S_{BS} comprising ROH and M_{B}OR is taken off at the lower end of RR_{B} and a vapour stream S_{BB} comprising water and ROH is taken off at the upper end of RR_{B},
(b) the vapour stream S_{AB}, and, when step (a2) is carried out, the vapour stream S_{BB}, is/are mixed with S_{AB} or, separately from S_{AB}, fed into a rectification column RD_{A} so that a mixture G_{A} comprising water and ROH is present in RD_{A},
(c) G_{A} is separated in RD_{A} into a vapour stream S_{DAB} comprising ROH at the upper end of RD_{A} and a bottom stream S_{DAS} comprising water and ROH at the lower end of RD_{A},
wherein the bottom stream S_{DAS} is discharged from RD_{A} at an offtake point E_{AS} at the lower end of RD_{A} and the vapour stream S_{DAB} is discharged from RD_{A} at an offtake point E_{AK} at the upper end of RD_{A},
(d) optionally, S_{DAS} is, in its entirety or partly, separated in at least one rectification column RD_{X} which is different from RD_{A}, and in which a mixture G_{X} comprising water and ROH is present, into a vapour stream S_{XB} comprising ROH at the upper end of RD_{X} and a bottom stream S_{XS} comprising water and optionally ROH at the lower end of RD_{X},
wherein the bottom stream S_{XS} is discharged from the at least one rectification column RD_{X} at an offtake point E_{XS} at the lower end of this column and the vapour stream S_{XB} is discharged from the at least one rectification column RD_{X} at an offtake point E_{XK} at the upper end of this column,
(e) energy is transferred from a heating vapour H₁ having a pressure p₁ to at least one component Q₁, as a result of which the heating vapour H₁ condenses at least partially and a condensate K₁ is thus obtained,
wherein:
(f) K₁ is at least partially depressurized to give a heating vapour H₂ having a pressure p₂, where p₂<p₁,
(g) H₂ is mixed with further heating vapour H₃ having a pressure p₃ so as to give a heating vapour H₄ having a pressure p₄, where p₂<p₄<p₃,
(h) energy is transferred from H₄ to at least one component Q₂,
wherein:
i) Q₁ and Q₂ are identical or different from one another,
ii) Q₁ and Q₂ are selected from the group consisting of S_{AE1}, S_{AE2}, RP_{A}, S_{AS}, S_{AB}, G_{A}, S_{DAS}, S_{DAB},
iii) Q₁ and Q₂ can, when step (a2) is carried out, alternatively or additionally be selected from the group consisting of S_{BE1}, S_{BE2}, RP_{B}, S_{BS}, S_{BB},
iv) Q₁ and Q₂ can, when step (d) is carried out, alternatively or additionally be selected from the group consisting of G_{X}, S_{XB}, S_{XS}.

17. The process of claim 16, wherein the heating vapour H₁ is contacted with the at least one component Q₁ so that energy is transferred from H₁ to the at least one component Q₁ and the heating vapour H₄ is contacted with the at least one component Q₂ so that energy is transferred from H₄ to the at least one component Q₂.

18. The process of claim 16, wherein energy is transferred from heating vapour H₁ to a heat transfer medium W₁ and the energy of W₁ is subsequently transferred to the at least one component Q₁ and energy from heating vapour H₄ is transferred to W₁ and energy is subsequently transferred from W₁ to the at least one component Q₂, where W₁ is none of S_{AE1}, S_{AE2}, RP_{A}, S_{AS}, S_{AB}, G_{A}, S_{DAS}, S_{DAB}, S_{BE1}, S_{BE2}, RP_{B}, S_{BS}, S_{BB}, G_{X}, S_{XB}, S_{XS}.

19. The process of claim 18, wherein W₁ comprises essentially water and/or alcohol ROH.

20. The process of claim 19, wherein the transfer of energy from W₁ to the at least one component Q₁ or Q₂ occurs by W₁ being mixed at least partly with Q₁ or Q₂ after energy has been transferred from heating vapour H₁ or H₄ to W₁.

21. The process of claim 16, wherein R is selected from methyl or ethyl.

22. The process of claim 16, wherein p₁<p₃.

23. The process of claim 16, wherein Q₁ and Q₂ are identical.

24. The process of claim 16, wherein the heating vapour H₄ is at least partially condensed so as to give a condensate K₂ by means of which a further step (f) in which the condensate K₂ is then used as condensate K₁ is carried out after step (h).

25. The process of claim 16, wherein the ratio of p₁/p₄ is in the range from 0.5 to 1.5.

26. The process of claim 16, wherein at least part of the vapour stream S_{AB} taken off at the offtake point E_{AK} at the upper end of the rectification column RD_{A} is fed to the reaction column RR_{A} at least as part of the feed stream S_{AE1} and, in cases in which step (a2) is carried out in the process of the invention, is alternatively or additionally fed to the reaction column RR_{B} at least as part of the feed stream S_{BE1}.

27. The process of claim 16, wherein step (d) is carried out.

28. The process of claim 27, wherein at least part of the vapour stream S_{XB} taken off at the offtake point E_{XK} at the upper end of the at least one rectification column RD_{X} is fed to the reaction column RR_{A} at least as part of the feed stream S_{AE1} and, in cases in which step (a2) is carried out in the process of the invention, is alternatively or additionally fed to the reaction column RR_{B} at least as part of the feed stream S_{BE1}.

29. The process of claim 16, wherein step (a2) is carried out.

30. The process of claim 16, which is carried out continuously.

31. The process of claim 20, wherein R is selected from methyl or ethyl.

32. The process of claim 21, wherein p₁<p₃.

33. The process of claim 22, wherein Q₁ and Q₂ are identical.

34. The process of claim 23, wherein the heating vapour H₄ is at least partially condensed so as to give a condensate K₂ by means of which a further step (f) in which the condensate K₂ is then used as condensate K₁ is carried out after step (h).

35. The process of claim 24, wherein the ratio of p₁/p₄ is in the range from 0.5 to 1.5.
